SEQUENCE LISTING (22 sequences)

SEQ ID NO: 1 - DNA, 2760 bp, Clarkia breweri
agaaaaccaaaccaccttaaacaagacaaccatgcagctcataacaaatttctcctcatcatcatcagaattgcagtttcttgtggataaggttaagagagaatcattgtcttcttcatcatctaatactcagaatttgtttctctcaacttcaccttatgacactgcttggctcgcccttatccctcatcctcatcatcaccatcaccatggccgacccatgtttgaaaaatgtctgcaatggattctccataaccagacaccacaaggtttctgggcagcagctggtgacaatatttccgacaccgacgatgacgtcaccctggattgtcttctatcaaccttggcttgcttagttgcactcaaaaggtggcagcttgctcccgacatgattcataaaggattggaatttgtaaatagaaacacagagagacttgtaatgaagcagaagccgagcgacgttcctcgttggttcaccatcatgttcccggcgatgctcgagcttgccggagcttccagtctccgagtcgatttcagcgagaatcttaacagaatcttggtggaactatctcaaaatagggatgatattctcacaagggaggaagttgatgagaagaagcaatactcaccattgctactatttctagaagcattgcctgcacaatcctatgacaatgatgttctaaagcaaattatagacaagaacttgagcaatgatggttctttattgcaatcgccttctgctacagcaagagcatacatgataacaggaaataccagatgcttatcgtatctacactctttaacaaatagctgctctaatggaggagtaccatcattctatcctgttgacgacgacctccatgatcttgtcatggtgaatcaactgacaaggtcgggtttgactgaacatctcatcccggagattgaccaccttctactcaaagttcaaaagaactacaaatacaaaaaagcatcaccaaaatcattgtatagcattgctgcggaactatacagggattcattagcattttggttgcttcgagtcaataatcactgggtatcaccatcaattttttgttggtttttagatgacgacgaaatccgtgatcacatcgaaacaaactacgaggaatttgctgccgtgcttcttaatgtgtatcgagctaccgatcttatgttctccggcgaagtccaacttgtcgaagcaagatctttcgctaccaagaatcttgagaaaatattagcaacaggaaacatacataaaactaatgcagatatctcatctagtttgcataagatgatcgaacacgaactaagagttccttggaccgcaagaatggaccatgttgaaaatcgaatttggatcgaagaaatagcttccagtgctttatggtttggaaaatcatcctaccttaggttatcttgctttcacaagatgagtttacagcaactcgcggtgaaaaattatacgcttcgacaattggtttaccgagacgagcttgcggaagttgagaggtggtctaaagaaagagggctatgtgacatgggattttgtagagagaaaaccgggtattgttactacgcatttgcggcaagtacttgtctgccgtggagttccgacgtgaggctggtcctgaccaaggcggcagttgtcattacagtggccgatgatttctttgatgtcgaaggatctatggttgatctcgaaaaattaacggatgcagttcggaggtgggatgcggaagggttaggcagccacagcaagacaatatttgaagccctggatgatcttgtaaatgaagttagactcaagtgtttccaacaaaatggacaagacatcaaaaacaatctccaacaattatggtatgaaacattccattcatggcttatggaagctaagtggggaaaggggttaacaagtaaaccatctgtagatgtgtatcttggaaatgcaatgacatccatagcagctcacaccatggtccttacagcatcctgtcttctaggtcccggtttcccggttcaccaactatggtcgcaaaggcgccaccaggacattacatccttgctcatggtcttgactcgcttgctaaatgacattcaatcctacttgaaagaagaagacgaaggaaaaataaactatgtatggatgtacatgatcgagaacaatcaagcgtcgatagatgactcggttcgacacgtccagacgataatcaatgtaaaaaagcaagaattcatccaacgtgttctatcggatcaacattgcaatctcccaaagtcattcaagcagctccatttctcctgcctcaaagtattcaacatgttcttcaactcctccaacattttcgacactgataccgaccttcttcttgacattcacgaagcttttgtttctccaccacaagttcccaaattcaaaccccacatcaagccacctcatcagcttccagcaacacttcagccacctcatcagccccaacaaataatggtcaataagaagaaggtggaaatggtttacaaaagctatcatcatccattcaaggttttcaccttgcagaagaaacaaagttcgggacatggtacaatgaatccaagggctagtatcttagcaggacccaacatcaaactatgtttcagttaacgaatacactaccttgttattagaagatgtcaccagtttccaaactcatctgctatgtatttacatatcatgtgataagcaaaattctctaataatctatccttttttatgtcaaa

SEQ ID NO: 2 - protein, 870 aa, Clarkia breweri
MQLITNFSSSSSELQFLVDKVKRESLSSSSSNTQNLFLSTSPYDTAWLALIPHPHHHHHHGRPMFEKCLQWILHNQTPQGFWAAAGDNISDTDDDVTLDCLLSTLACLVALKRWQLAPDMIHKGLEFVNRNTERLVMKQKPSDVPRWFTIMFPAMLELAGASSLRVDFSENLNRILVELSQNRDDILTREEVDEKKQYSPLLLFLEALPAQSYDNDVLKQIIDKNLSNDGSLLQSPSATARAYMITGNTRCLSYLHSLTNSCSNGGVPSFYPVDDDLHDLVMVNQLTRSGLTEHLIPEIDHLLLKVQKNYKYKKASPKSLYSIAAELYRDSLAFWLLRVNNHWVSPSIFCWFLDDDEIRDHIETNYEEFAAVLLNVYRATDLMFSGEVQLVEARSFATKNLEKILATGNIHKTNADISSSLHKMIEHELRVPWTARMDHVENRIWIEEIASSALWFGKSSYLRLSCFHKMSLQQLAVKNYTLRQLVYRDELAEVERWSKERGLCDMGFCREKTGYCYYAFAASTCLPWSSDVRLVLTKAAVVITVADDFFDVEGSMVDLEKLTDAVRRWDAEGLGSHSKTIFEALDDLVNEVRLKCFQQNGQDIKNNLQQLWYETFHSWLMEAKWGKGLTSKPSVDVYLGNAMTSIAAHTMVLTASCLLGPGFPVHQLWSQRRHQDITSLLMVLTRLLNDIQSYLKEEDEGKINYVWMYMIENNQASIDDSVRHVQTIINVKKQEFIQRVLSDQHCNLPKSFKQLHFSCLKVFNMFFNSSNIFDTDTDLLLDIHEAFVSPPQVPKFKPHIKPPHQLPATLQPPHQPQQIMVNKKKVEMVYKSYHHPFKVFTLQKKQSSGHGTMNPRASILAGPNIKLCFS

SEQ ID NO: 3 - DNA, 2170 bp, Mentha spicata
agagagagagaggaaggaaagattaatcatggctctcaaagtgttaagtgttgcaactcaaatggcgattcctagcaacctaacgacatgtcttcaaccctcacacttcaaatcttctccaaaactgttatctagcactaacagtagtagtcggtctcgcctccgtgtgtattgctcctcctcgcaactcactactgaaagacgatccggaaactacaacccttctcgttgggatgtcaacttcatccaatcgcttctcagtgactataaggaggacaaacacgtgattagggcttctgagctggtcactttggtgaagatggaactggagaaagaaacggatcaaattcgacaacttgagttgatcgatgacttgcagaggatggggctgtccgatcatttccaaaatgagttcaaagaaatcttgtcctctatatatctcgaccatcactattacaagaacccttttccaaaagaagaaagggatctctactccacatctcttgcatttaggctcctcagagaacatggttttcaagtcgcacaagaggtattcgatagtttcaagaacgaggagggtgagttcaaagaaagccttagcgacgacaccagaggattgttgcaactgtatgaagcttcctttctgttgacggaaggcgaaaccacgctcgagtcagcgagggaattcgccaccaaatttttggaggaaaaagtgaacgagggtggtgttgatggcgaccttttaacaagaatcgcatattctttggacatccctcttcattggaggattaaaaggccaaatgcacctgtgtggatcgaatggtataggaagaggcccgacatgaatccagtagtgttggagcttgccatactcgacttaaatattgttcaagcacaatttcaagaagagctcaaagaatccttcaggtggtggagaaatactgggtttgttgagaagctgcccttcgcaagggatagactggtggaatgctacttttggaatactgggatcatcgagccacgtcagcatgcaagtgcaaggataatgatgggcaaagtcaacgctctgattacggtgatcgatgatatttatgatgtctatggcaccttagaagaactcgaacaattcactgacctcattcgaagatgggatataaactcaatcgaccaacttcccgattacatgcaactgtgctttcttgcactcaacaacttcgtcgatgatacatcgtacgatgttatgaaggagaaaggcgtcaacgttataccctacctgcggcaatcgtgggttgatttggcggataagtatatggtagaggcacggtggttctacggcgggcacaaaccaagtttggaagagtatttggagaactcatggcagtcgataagtgggccctgtatgttaacgcacatattcttccgagtaacagattcgttcacaaaggagaccgtcgacagtttgtacaaataccacgatttagttcgttggtcatccttcgttctgcggcttgctgatgatttgggaacctcggtggaagaggtgagcagaggggatgtgccgaaatcacttcagtgctacatgagtgactacaatgcatcggaggcggaggcgcggaagcacgtgaaatggctgatagcggaggtgtggaagaagatgaatgcggagagggtgtcgaaggattctccattcggcaaagattttataggatgtgcagttgatttaggaaggatggcgcagttgatgtaccataatggagatgggcacggcacacaacaccctattatacatcaacaaatgaccagaaccttattcgagccctttgcatgagagatgatgacgagccatcgtttacttacttaaattctaccaaagtttttcgaaggcatagttcgtaatttttcaagcaccaataaataaggagaatcggctcaaacaaacgtggcatttgccaccacgtgagcacaagggagagtctgtcgtcgtttatggatgaactattcaatttttatgcatgtaataattaagttcaagttcaagagccttctgcatatttaactatgtatttgaatttatcgagtgtgattttctgtctttggcaacatatatttttgtcatatgtggcatcttattatgatatcatacagtgtttatggatgatatgatactatc

SEQ ID NO: 4 - protein, 599 aa, Mentha spicata
MALKVLSVATQMAIPSNLTTCLQPSHFKSSPKLLSSTNSSSRSRLRVYCSSSQLTTERRSGNYNPSRWDVNFIQSLLSDYKEDKHVIRASELVTLVKMELEKETDQIRQLELIDDLQRMGLSDHFQNEFKEILSSIYLDHHYYKNPFPKEERDLYSTSLAFRLLREHGFQVAQEVFDSFKNEEGEFKESLSDDTRGLLQLYEASFLLTEGETTLESAREFATKFLEEKVNEGGVDGDLLTRIAYSLDIPLHWRIKRPNAPVWIEWYRKRPDMNPVVLELAILDLNIVQAQFQEELKESFRWWRNTGFVEKLPFARDRLVECYFWNTGIIEPRQHASARIMMGKVNALITVIDDIYDVYGTLEELEQFTDLIRRWDINSIDQLPDYMQLCFLALNNFVDDTSYDVMKEKGVNVIPYLRQSWVDLADKYMVEARWFYGGHKPSLEEYLENSWQSISGPCMLTHIFFRVTDSFTKETVDSLYKYHDLVRWSSFVLRLADDLGTSVEEVSRGDVPKSLQCYMSDYNASEAEARKHVKWLIAEVWKKMNAERVSKDSPFGKDFIGCAVDLGRMAQLMYHNGDGHGTQHPIIHQQMTRTLFEPFA

SEQ ID NO: 5 - DNA, 1912 bp, Salvia officinalis
agcaatattacaactaacaataaaaatgtcttccattagcataaacatagctatgccactgaattccctccacaactttgagaggaaaccttcaaaagcatggtctacctcttgcactgcacccgcagctcgcctccgggcatcttcctccttacaacaagaaaaacctcaccaaatccgacgctctggggattaccaaccctctctttgggatttcaattacatacagtctctcaacactccgtataaggagcagagacactttaataggcaagcagagttgattatgcaagtgaggatgttgctcaaggtaaagatggaggcaattcaacagttggagttgattgatgacttgcaatacctgggactgtcttatttctttcaagatgagattaaacaaatcttaagttctatacacaatgagcccagatatttccacaataatgatttgtatttcacagctcttggattcagaatcctcagacaacatggttttaatgtttccgaagatgtatttgattgtttcaaaattgagaagtgcagtgatttcaatgcaaaccttgctcaagatacgaagggaatgttacaactttatgaagcatctttccttttgagagaaggtgaagatacattggagctagcaagacgattttccaccagatctctacgagaaaaatttgatgaaggtggtgatgaaattgatgaagatctatcatcgtggattcgccattccttggatcttcctcttcattggagggtccaaggattagaggcaagatggttcttagatgcttatgcgaggaggccggacatgaatccacttattttcaaactcgccaaactcaacttcaatattgttcaggcaacatatcaagaagaactgaaagatatctcaaggtggtggaatagttcgtgccttgctgagaaactcccatttgtgagagataggattgtggaatgcttcttttgggccatcgcggcttttgagcctcaccaatatagttatcagagaaaaatggccgccgttattattactttcataacaattatcgatgatgtttatgatgtgtatggaacaatagaagaactagaactattaacagatatgattcgcagatgggataataaatcaataagccaacttccatattatatgcaagtgtgctatttggcactatacaacttcgtttctgagcgggcttacgatattctaaaagatcaacatttcaacagcatcccatatttacagagatcgtgggtaagtttggttgaaggatatcttaaggaggcatactggtactacaatggctataaaccaagcttggaagaatatctcaacaacgccaagatttcaatatcggctcctacaatcatatcccagctttattttacattagcaaactcgattgatgaaacagctatcgagagcttgtaccaatatcataacatactttacctatcaggaaccatattaaggcttgctgacgatcttgggacatcacaacatgagctggagagaggagacgtaccgaaagcaatccagtgctacatgaatgacacaaatgcttcggagagagaggcggtggaacacgtgaagtttctgataagggaggcgtggaaggagatgaacacggtcacaacagccagcgattgtccgtttacggatgatttggttgcggccgcagctaatcttgcaagggcggctcagtttatatatctcgacggggatgggcatggcgtgcaacactcagaaatacatcaacagatgggaggcctgctattccagccttatgtctgaataaatcgaaaatccaacctactatgtatccctcgataatatattcttggggttaacatgtttaattaaagttctaattdaaagagctgaatcgatcctcaaaaaaaaaaaaaa

SEQ ID NO: 6 - protein, 590 aa, Salvia officinalis
MSSISINIAMPLNSLHNFERKPSKAWSTSCTAPAARLRASSSLQQEKPHQIRRSGDYQPSLWDFNYIQSLNTPYKEQRHFNRQAELIMQVRMLLKVKMEAIQQLELIDDLQYLGLSYFFQDEIKQILSSIHNEPRYFHNNDLYFTALGFRILRQHGFNVSEDVFDCFKIEKCSDFNANLAQDTKGMLQLYEASFLLREGEDTLELARRFSTRSLREKFDEGGDEIDEDLSSWIRHSLDLPLHWRVQGLEARWFLDAYARRPDMNPLIFKLAKLNFNIVQATYQEELKDISRWWNSSCLAEKLPFVRDRIVECFFWAIAAFEPHQYSYQRKMAAVIITFITIIDDVYDVYGTIEELELLTDMIRRWDNKSISQLPYYMQVCYLALYNFVSERAYDILKDQHFNSIPYLQRSWVSLVEGYLKEAYWYYNGYKPSLEEYLNNAKISISAPTIISQLYFTLANSIDETAIESLYQYHNILYLSGTILRLADDLGTSQHELERGDVPKAIQCYMNDTNASEREAVEHVKFLIREAWKEMNTVTTASDCPFTDDLVAAAANLARAAQFIYLDGDGHGVQHSEIHQQMGGLLFQPYV

SEQ ID NO: 7 - DNA, 1564 bp, Clarkia breweri
atttatttcacttccaattacataagcaaacactctgctgcttttgtctgtcttatcattttccttataacacccctcaaacaaaatacccttgaaaccctagctaggttacacgatgaatgttacgatgcactccaagaagttacttaaaccatctattcccaccccaaatcaccttcaaaagttgaacttgtcattgctagatcaaattcagatccccttctacgtgggattgatctttcactacgaaaccttatctgacaactccgatattaccctttccaaacttgagagctccctctccgaaaccctaaccctatattaccatgtggccgggaggtataatggaaccgattgtgtgatcgaatgcaatgaccaaggcatcgggtatgtagaaacagcatttgatgttgaactacatcaatttcttttgggagaagaatccaataatctcgacttgcttgtcgggttgtcgggattcttgtccgagactgagactccgccccttgctgctattcaactcaatatgttcaagtgcggcgggttagttatcggagcacagttcaaccatattataggagacatgttcacaatgtctaccttcatgaactcatgggccaaagcttgccgtgtcggcatcaaagaggtcgctcatccaactttcgggttggcgcctctcatgccttctgcaaaggtactaaatattcccccgccaccttccttcgaaggagtgaaatttgtgtccaagagattcgttttcaatgaaaacgcaataacacgactaagaaaagaagctaccgaagaagatggtgatggtgatgatgatcagaagaagaagcgcccttcacgagtcgacctagtaaccgcatttttgtccaaaagcctaatcgagatggattgtgccaaaaaagagcagactaaaagccgaccatctttaatggtacacatgatgaacttacgtaagagaacaaaactagcattggaaaacgatgttagcggtaatttcttcattgtagtaaatgcagagtccaaaataacggttgcaccaaagataactgacttaaccgaatcactgggcagtgcatgtggtgaaataattagtgaagtagcaaaagttgatgatgcggaggtggtaagttctatggtgctgaattcagtaagagagttttattatgaatgggggaaaggtgaaaagaatgtatttttgtatactagctggtgcagatttccattgtacgaggttgactttgggtgggggatacccagcttagttgacactactgctgttccatttgggttgattgttctaatggatgaagcgccggcaggagatggaattgcagttcgtgcatgcttaagtgagcatgacatgattcaattccaacaacaccaccaactgctttcatatgtttcctaaatacttatatattattattatatatattggttaagagctatttgtttggctgttgctatctttttttttttcttcctagtaaattaagtgttatcgtattaattatatgcttgttgtggcttgttcatacacgtgtgcatatttttt

SEQ ID NO: 8 - protein, 433 aa, Clarkia breweri
MNVTMHSKKLLKPSIPTPNHLQKLNLSLLDQIQIPFYVGLIFHYETLSDNSDITLSKLESSLSETLTLYYHVAGRYNGTDCVIECNDQGIGYVETAFDVELHQFLLGEESNNLDLLVGLSGFLSETETPPLAAIQLNMFKCGGLVIGAQFNHIIGDMFTMSTFMNSWAKACRVGIKEVAHPTFGLAPLMPSAKVLNIPPPPSFEGVKFVSKRFVFNENAITRLRKEATEEDGDGDDDQKKKRPSRVDLVTAFLSKSLIEMDCAKKEQTKSRPSLMVHMMNLRKRTKLALENDVSGNFFIVVNAESKITVAPKITDLTESLGSACGEIISEVAKVDDAEVVSSMVLNSVREFYYEWGKGEKNVFLYTSWCRFPLYEVDFGWGIPSLVDTTAVPFGLIVLMDEAPAGDGIAVRACLSEHDMIQFQQHHQLLSYVS

SEQ ID NO: 9 - DNA, 1321 bp, Clarkia breweri
gcggacgaggcattagtcgcagtcggaacatatatacgtttcccttataaataatggaggtaataatgcaaggtgcaaaactcaactagaagaagaagaagaatggatgtacggcaagttcttcacatgaagggtggcgccggagaaaatagttatgctatgaactcatttattcagagacaagtgatatccatcacaaaacccataactgaggcggccatcactgccctttactccggcgacactgttacgacaaggctcgccatagccgatttaggatgttcatctgggccgaacgcattatttgcagtgaccgaactgatcaaaactgtagaagagctacgtaagaagatgggacgagaaaactcgccggagtaccaaatattcttgaatgatcttcccggaaatgactttaatgctatatttaggtctttgccgattgaaaacgacgtcgatggagtttgctttatcaatggtgttcctggttccttctatggcaggcttttccctagaaataccctacactttattcattcttcatatagcctcatgtggctatctcaggttcctataggaatagaaagcaacaaggggaatatatacatggcaaatacttgcccacaaagtgtcctcaatgcttactacaagcaattccaggaagaccatgcgttgtttctcaggtgtcgagctcaagaagtagtgccaggtggacgcatggtgttgacaattctaggaagacgaagtgaggatcgagctagcactgaatgctgtctcatttggcaactcttagcgatggctctcaatcagatggtttctgagggactaatagaagaagagaagatggataagttcaacattcctcagtatacaccatctccaacagaagtagaagcagagatcctaaaagaagggtcttttttgattgaccatatagaggcttcagaaatatactggagtagctgcactaaagatggtgatggtggtgggtctgttgaggaagaaggttacaacgtggctcggtgcatgagagcagtggccgagccattgctgctcgaccattttggtgaagccatcattgaagatgtgttccataggtataaactactcataatcgaaagaatgtctaaagagaagaccaaattcatcaacgtcattgtctctctcattcgaaaatcagattaattcatccatatggtcggcaaattaattcagtcgatcaatataattatgatgggactttatatacttgctatatatatagtattagaatgattttttttttttttggttgaaaaagtgaattgcaagtaataaaagtgta

SEQ ID NO: 10 - protein, 359 aa, Clarkia breweri
MDVRQVLHMKGGAGENSYAMNSFIQRQVISITKPITEAAITALYSGDTVTTRLAIADLGCSSGPNALFAVTELIKTVEELRKKMGRENSPEYQIFLNDLPGNDFNAIFRSLPIENDVDGVCFINGVPGSFYGRLFPRNTLHFIHSSYSLMWLSQVPIGIESNKGNIYMANTCPQSVLNAYYKQFQEDHALFLRCRAQEVVPGGRMVLTILGRRSEDRASTECCLIWQLLAMALNQMVSEGLIEEEKMDKFNIPQYTPSPTEVEAEILKEGSFLIDHIEASEIYWSSCTKDGDGGGSVEEEGYNVARCMRAVAEPLLLDHFGEAIIEDVFHRYKLLIIERMSKEKTKFINVIVSLIRKSD

SEQ ID NO: 11 - DNA, 1486 bp, Clarkia breweri
ataagtaccagaaagctctcataacagaaaaaaaaaaaaaaaatgggatctaccggaaatgcagagatccagataatccccacccactcctccgacgaggaagccaacctcttcgccatgcagctggccagcgccgccgttctccccatggcccttaaggccgccatcgagctcgacgtccttgagatcatggccaagtccgtccctcccagcggctacatctctccggcggagattgccgcgcagcttcctaccaccaaccctgaagctccggtgatgcttgaccgtgtcctccgcctcctagccagctactccgtcgtaacatacactctccgggaacttcccagcggcaaggtggagaggctgtacggcctcgcccctgtctgcaagttcttgaccaagaacgaggatggagtttctcttgctccttttttgctcacggctaccgacaaggtccttttggagccctggttttacttgaaagatgcgattcttgaaggaggaattccattcaataaagcgtatggaatgaatgaattcgattaccatggaacagaccacagattcaacaaggtgttcaacaagggaatgtccagcaactctaccatcaccatgaagaagatccttgaaatgtacaacggattcgaggggctaacaacgattgtcgatgttgggggcggtacaggtgccgtggctagcatgattgttgctaagtatccttccatcaacgccatcaacttcgacctgcctcacgttattcaggatgctccagctttttctggtgttgaacatcttggaggagatatgtttgatggcgtacccaaaggcgacgctatattcatcaagtggatttgccacgactggagcgatgagcattgcctgaagttgctgaaaaactgctatgctgcacttcccgaccatggcaaggtcattgttgcagaatacatccttcctccgtctcctgacccgagtatcgccaccaaggtagtcatccataccgacgccctcatgttggcctacaacccaggcggcaaagaaaggactgagaaggagttccaggctttggctatggcttccggattcaggggtttcaaagtagcatcttgtgccttcaacacttacgtcatggagttcctcaaaaccgcgtaaatgattatgttcgaaaccgaccaattgtgaatggctgcaaaactattcctatcgaataagtgagttttatgctggttgttgctgaatatatcagtatgcaagagtatgctcttccaataaatcttagaatagtagtgactttgtacaagtcctagaatagtggtaagctgtgtctttactgttaaaagtttgtcgtatggccactataaaaggaaagtatctgcgtctttgttgtaattagcaattcactgtagctgagatcctcccctcagcttaggtgtttgctctcaattattctccagcttaatgtgaattgagcctgactggagcttattag

SEQ ID NO: 12 - protein, 368 aa, Clarkia breweri
MGSTGNAEIQIIPTHSSDEEANLFAMQLASAAVLPMALKAAIELDVLEIMAKSVPPSGYISPAEIAAQLPTTNPEAPVMLDRVLRLLASYSVVTYTLRELPSGKVERLYGLAPVCKFLTKNEDGVSLAPFLLTATDKVLLEPWFYLKDAILEGGIPFNKAYGMNEFDYHGTDHRFNKVFNKGMSSNSTITMKKILEMYNGFEGLTTIVDVGGGTGAVASMIVAKYPSINAINFDLPHVIQDAPAFSGVEHLGGDMFDGVPKGDAIFIKWICHDWSDEHCLKLLKNCYAALPDHGKVIVAEYILPPSPDPSIATKVVIHTDALMLAYNPGGKERTEKEFQALAMASGFRGFKVASCAFNTYVMEFLKTA

SEQ ID NO: 13 - DNA, 1363 bp, Antirrhinum majus
gccggacgccaaagaaaaatgaaagtgatgaagaaacttttgtgtatgaatattgcaggagatggtgaaactagctacgccaacaattctggccttcaaaaagttatgatgtcaaaatcattgcatgttttagacgaaacccttaaagatattatcggtgatcatgttggcttcccaaaatgcttcaagatgatggatatgggttgttcatcagggcctaacgcccttttggtcatgtccggcattataaatacaattgaggatttgtacacagagaagaatattaatgaattacctgaatttgaggtttttctgaacgatcttccagacaacgacttcaacaacctcttcaaattgttatcacatgagaatggaaactgctttgtatatggtttgcctggatctttctacgggagactattgccaaaaaagagcctacactttgcttattcttcctacagtattcactggctctctcaggttcctgaagggctggaggataataacagacaaaacatttacatggcaacggaaagtcctccggaagtgtacaaagcatacgcaaagcaatacgaaagagacttctccacatttctaaagttgcgaggcgaggaaattgtaccaggtggacgcatggtcttgacatttaacggcagaagtgttgaagatccctcgagcaaagatgacttagcaattttcacattgcttgcaaaaacactagttgatatggtggctgaggggcttgtcaagatggacgatttgtactcgtttaacattcctatttactcaccatgtacgcgcgaagtagaggcagcaattctgagtgaagggtcttttacgttggacaggctagaggtctttcgtgtttgttgggatgcaagtgactacacagatgacgatgatcagcaagacccatcaatctttggcaaacaaaggagtggaaaatttgtggcagattgtgtacgggctattacggaaccaatgctggctagccattttgggagcactattatggatcttctatttggaaagtatgcaaagaaaatagtggagcatctatctgtggagaactcgtcatatttcagcatagtagtttctctaagtaggagatgaagtcaacaggatggagataccacgtatttcggcacatttgctgtaaaatgatgatataattatagaataaaattatattgaatgcagaataattgtgtcgcacaccattgtttccaatactatctacatgcaattgttaattcagtttttgattttgcttcttctctttctaatactgttcttttgttgcagaggtgtgaactgatcagcacctatatatagtactatttttatagcagaagtaatggaa

SEQ ID NO: 14 - protein, 364 aa, Antirrhinum majus
MKVMKKLLCMNIAGDGETSYANNSGLQKVMMSKSLHVLDETLKDIIGDHVGFPKCFKMMDMGCSSGPNALLVMSGIINTIEDLYTEKNINELPEFEVFLNDLPDNDFNNLFKLLSHENGNCFVYGLPGSFYGRLLPKKSLHFAYSSYSIHWLSQVPEGLEDNNRQNIYMATESPPEVYKAYAKQYERDFSTFLKLRGEEIVPGGRMVLTFNGRSVEDPSSKDDLAIFTLLAKTLVDMVAEGLVKMDDLYSFNIPIYSPCTREVEAAILSEGSFTLDRLEVFRVCWDASDYTDDDDQQDPSIFGKQRSGKFVADCVRAITEPMLASHFGSTIMDLLFGKYAKKIVEHLSVENSSYFSIVVSLSRR

SEQ ID NO: 15 - protein, 4 aa, Zucchini yellow mosaic virus
FRNK

SEQ ID NO: 16 - protein, 4 aa, Artificial sequence
  Zucchini yellow mosaic virus, HC protein mutated
FINK

SEQ ID NO: 17 - protein, 3 aa, Zucchini yellow mosaic virus
DAG

SEQ ID NO: 18 - protein, 3 aa, Artificial sequence
  Zucchini yellow mosaic virus, CP protein mutated
DTG

SEQ ID NO: 19 - protein, 4 aa, Zucchini yellow mosaic virus
KLSC

SEQ ID NO: 20 - protein, 4 aa, Artificial sequence
  Zucchini yellow mosaic virus, HC protein mutated
ELSC

SEQ ID NO: 21 - protein, 3 aa, Zucchini yellow mosaic virus
PTK

SEQ ID NO: 22 - protein, 3 aa, Artificial sequence
  Zucchini yellow mosaic virus, HC protein mutated
PAK

(19) United States
(12) Patent Application Publication
(10) Pub. No.: US 2004/0078847 A1
(43) Pub. Date: Apr. 22, 2004

(54) Method of enhancing entomophilous

(76) Inventor: Nitzan Paldi, D.N. Ha'Ela (IL)

Correspondence Address: Anthony Castorina, G E Ehrlich, Suite 207, 2001 Jefferson Davis Highway, Arlington, VA, 22202, US

(21) Appl. No.: 10/469,993

(22) PCT Filed: Feb. 24, 2002
(86) PCT No.: PCT/IL02/00142

(51) Int. Cl. A01H 1/00; C12N 15/82

(52) U.S. Cl. 800/284; 800/287

(57) ABSTRACT

A method of enhancing insect assisted cross-pollination between flowering plants of a single plant species, the flowering plants being of at least two different genetic backgrounds (e.g., different cultivars). The method is effected by co-expressing in plants of the at least two different genetic backgrounds at least one scent biosynthetic enzyme and growing the plants in a cross-pollination vicinity in a presence of at least one pollinating insect.

### FIELD AND BACKGROUND OF THE INVENTION

The present invention relates to a method of enhancing entomophilous assisted cross-pollination and, more particularly, to a method of enhancing entomophilous assisted cross-pollination between flowers of cross-fertilizing cultivars or genotypes, such as parental genotypes of plants used for the production of hybrid seeds, via co-expression of scent producing enzymes.

Entomophilous Pollination

Entomophilous pollination of crops is a common phenomenon. Honeybees, for example, are hired for pollination worldwide, and over 2 million hives are used every year in the United States alone for pollinating crops such as sunflower, almonds, watermelon and many more. It has been estimated that the added value from pollination to crop yield is many times larger than the value of honey produced, and reaches at least $ 9.3 billion per annum in the U.S. alone (Robinson et al., 1989).

During evolution flowers evolved to regulate pollinator visits to such times when the insect facilitates successful fertilization. Thus, pollinator visits are increased when the stigma is receptive and the gametophyte sufficiently developed. To this end, flowers often reward potential pollinators with a high energy (nectar) or a high protein (pollen) reward. Such rewards are typically offered or maximize only at such times when a visitor pollinator would facilitate successful fertilization. Other rewards such as providing shelter are less common. Insects track down rich nectar sources and honeybees in particular are proficient at relaying this information to their colony (Seeley and Levien, 1985). In order to attract pollinators, the flower has to signal its readiness and activate interorgan regulation of signal-reward-compatibility in order to remain reliable in the course of evolution. The signal is relayed as a combination of visual and olfactory “messages”. These include pigment biosynthesis and emission of volatiles, both of which require the “expensive” triggering and utilization of unrelated secondary metabolite pathways. Recently it has been shown that pollinator-specific scents are produced in plants of different families. Examples include sweet smelling benzenoid esters for moths (Dudareva et al., 1998a), oligomethyl oligosulphides for flies from the Sarcophagaceae (Borg-Karlson et al., 1994a) and for rain-forest bats (Bestmann et al., 1997), and the extreme adaptation of orchids to pheromone-specific signals of bees (Schiestl et al., 1999). Different olfactory adaptations by flowers may occur even within plant genera and in some cases even among ecotypes of the same species, possibly to adapt to different pollinators in different environments (Borg-Karlson et al., 1994b).

Reward too has been implicated to be pollinator-specific. Preferences of reducing versus non-reducing sugar in the nectar may differ between pollinators (Baker and Baker 1983), or secretion of primary and secondary metabolites such as amino acids and flavor compounds (Baker and Baker 1977). This is most probable since nectar has no role in the plant other than as a pollinator appeaser. Less attention has been focused on the correlation between pollen content and flower-insect co-adaptation since pollen germination and fertilization are independent of pollinator type and are flower specific.

Honeybee preferences for nectar production in volume and concentration and their relative influence on visits to flowers, has been studied prolifically and reviewed extensively (see, e.g., Widrelecher and Senechal, 1992). This and other studies show that there is a direct correlation between the amount of caloric energy provided by the flowers, and their subsequent attractability to bees.

Compatibility of pollen on the stigma, its germination, growth or its subsequent fusion with the gametophyte for the creation of the zygote, control inter-organ regulation of the cessation of signal and reward. A continuation of these signals after successful fertilization, has taken place, would constitute wastage of expensive secondary resources. Exceptions to this might be when a plant has many flowers and wishes to continue attracting insects even after some flowers from the plant were fertilized. Alternatively, compound fruits like Cucurbitaceae or Strawberry may require multiple pollination events for normal fruit development. Yet, if these signals continue after the flower's reward has been exhausted, insects will encounter non-rewarding flowers. In fact, successful pollination and pollen germination with subsequent fertilization eventually results in a regulated cascade of events culminating in a termination of both the visual and olfactory signals (O'neill et al., 1993).

The Flower Bouquet

Volatiles are produced in all parts of the flower in different relative abundance. In Clarkia Brewri, for example, the petals harbor most of the activity of the scent producing enzymes (Pichersky et al., 1994). Localization of specific scent to the pollenkitt enables pollinator discrimination of pollen rewarding versus non-rewarding flowers in, for example, the genus Rosa (Dobson et al., 1987, Dobson et al., 1996). It was previously assumed that glycosylases act on glycosilated precursors that are transported into the flower (Loughrin et al., 1992) and are “activated” when the flower opens (Watanabe et al., 1993). However recent data seems to refute this dogma and suggests an alternative whereby biosynthetic enzymes are active in the flower organs, where scent genes are differentially expressed (see, e.g., Dudareva et al., 1996, and a review by Dudareva et al., 1999). The time dependent manner of expression of these genes points to a common regulatory mechanism (Dudareva et al., 1998b). The checklist of volatiles produced by flowers is enormous (Knudsen et al., 1993) and ever growing.

If the emission of volatiles is to be manipulated in any way, it must be done with an appreciation of the external as well as endogenous factors influencing it. For example, different climatic conditions such as light intensity, humidity and irrigation affect volatile emission (Jackobson and Olsen, 1994), but temperature is the most pronounced factor (Hanstead et al., 1994). Diurnal circadian variations are also common with asynchronous emissions of the different constituents at different times (Loughrin et al., 1993, Nielsen et al., 1995). Most importantly, peak emissions of certain constituents often correlate with pollinator activity (Dudarareva et al., 1999).

Analyzing Volatile Emissions

Gas chromatography-Mass Spectronomy (GC-MS) is the state of the art method for analyzing volatile emissions. Since macerated and whole flowers emit qualitatively and quantitatively different aromas (Tollsten and Bergstrom 1988), it is necessary to make in-situ collections of volatiles directly from a living plant. The confounding problem of vegetative odor constituents may be circumvented by differential chromatograms of plants with or without flowers (Pellymer et al., 1987).

Attempts to Enhance Honeybee Visitation to Flowers

Attempts to attract bees to flowers, via spraying with sugar and/or synthetic Nasanov pheromone derivatives, in order to increase pollination, have proved altogether unsuccessful (Rapp et al., 1984, Elmsrom and Maynard, 1990, Shultheis et al., 1994, Ambrose 1995).

It seems that the above attempts were lacking in their capability to reliably attract bees to the flowers and facilitate enhanced pollination for the following reasons:

First, the spray was applied over the whole plant. Thus the ensuing odor does not emanate from the flower, which is probably a confounding factor for the bees.

Second, the spraying is done arbitrarily without taking into account the timing of nectar secretion, thus causing the bees to become averse to these odors which are associated with no reward (see section on associative learning in honeybees below).

Another approach, which probably involved the biggest project carried out in attempting at pollination enhancement, was to use mass spraying of honeybee Queen Mandibular Pheromone (QMP) directly on the flowering trees. The rationale behind the use of QMP is that foraging bees will return to the hive bearing QMP residue, and will thus attract more bees to their waggle dance (Currie et al., 1992a). However, this rationale disregards the fact that QMP is an elicitor of retinue behavior inside the hive for queen nursing bees (De-Hazan et al., 1989) and is thus completely context non-specific foraging behavior. Indeed, honeybee pheromones are unlikely to elicit any response when used out of context (Winston, 1995). Field trials, that involved the spraying of QMP on orchards in Canada, showed questionable statistical improvement of yield only in bad weather conditions and in one out of the two years through which the trials were conducted (Currie et al., 1992a, Currie et al., 1992b).

The Associative Learning Capabilities of Honeybees

The ability of honey bees, Apis mellifera L., to discriminate between differential rewards in natural settings is mostly based on assessment of the flower bouquet in relation to reward (Masson et al., 1993). Odors may either be innately attractive or repellent to the honey bees, sometimes as a function of their relative concentration and abundance (Henning et al., 1992), but mostly through their association to a more profitable nectar or pollen reward (Menzel 1993, Dobson et al., 1996). In this manner, honeybees can discriminate between different genotypes of the same species (Wolf et al., 1999) or between different flowering stages of a particular genotype (Pham-Delegue et al., 1989). Based on circadian, diurnal, temperature dependent or asynchronous emissions of flower odors (Loughrin et al., 1991, Loughrin et al., 1993, Hansted et al., 1994, Nielsen et al., 1995), honey bees learn to associate certain constituents of a bouquet with current reward (Blight et al., 1997).

The associative learning capability of honeybees has been extensively studied through the Proboscis Extension Reflex (PER) Paradigm (Bitterman et al., 1983, Menzel and Muller, 1996). In PER conditioning, bees are harnessed so that they can only freely move their mouthparts and antennae. Sucrose stimulation to the antennae serves as an unconditioned stimulus (US) and elicits proboscis extension as the unconditioned response (UR). If an odor as a conditioned stimulus (CS) is properly paired with the US, the odor itself becomes capable of eliciting proboscis extension as a conditioned response (CR). Many phenomena relating to the behavior of honeybees have been elucidated with this experimental paradigm. Some recent examples include blocking (Smith and Cobey, 1994, Hosler and Smith, 2000) factors influencing time-dependent memory formation (Hammer and Menzel, 1995, Fiala et al., 1999), preference of amino-acids in sucrose solution (Kim and Smith, 2000), sensory preconditioning (Muller et al., 2000), acquisition, extinction, and reversal learning (Smith, 1991, Scheiner et al., 1999), caste etiology (Ray and Ferneyhough, 1999), visual modulation and its relation to olfaction (Gerber and Smith, 1998), the effect of genotype on response thresholds to sucrose (Page et al., 1998) and odor intensity and its roles in discrimination, overshadowing and memory consolidation (Bhagavan et al., 1997, Pelz et al., 1997). However, most of these experiments have been performed only within the context of the PER reaction.

Some work has been recently done on elucidating associative learning in free flying honeybees. Jakobsen et al., (1995) found that honeybees, in contrast to bumblebees, disregarded positional cues for reward, and used odoriferous stimuli to locate a food source on a rotating arena. A recent replication and elaboration on work done by von-Frisch in 1919, demonstrated that free flying honeybees significantly distinguished between a vast majority of compound pairs bearing structural similarity to each other (Laska et al., 1999). In both these studies, conditioning was performed to sucrose reward against a background of a non-rewarding odor.

Only a few attempts have been made to test associative conditioning in multiple contexts. Gerber et al., (1996) found that bees that had previously foraged on Basswood florets could transfer their experience to the PER associative context, by extending their proboscis when presented with Basswood florets while restrained. Conversely, restrained bees that were conditioned to a floral odor, spent more time oriented towards that odor in a free walking olfactometer (Sandoz et al., 2000).

When restrained bees are conditioned to a specific odor, they sometimes generalize and extend their proboscis when confronted with a novel odorant; the level of generalization depends upon the structural similarity of the novel odorant to the conditioned one (Getz and Smith 1990). It seems that neural representations of unrelated odors are assigned different glomeruli (Joergus et al., 1997) whereas closely related compounds seem to be assigned to one glomerulus. Thus the ability to discriminate structural analogs requires a further dimension of temporal oscillatory synchronization (Stopfer et al., 1997), which is probably enhanced by modification of odor representation by associative learning (Faber et al., 1999). In the field, honey bees are able to discriminate even between closely related flowers and recognize which of these is most rewarding (Pham-Delegue et al., 1989). The bees often pick salient major components of the bouquet and disregard the other components in their associative acquisition of an odor-reward pairing (Blight et al., 1997, Le Metayer et al., 1997). This strategy saves the need to relate to each of the odors in the myriad of olfactory stimulations in the field. Separate analysis of components of a mixture, in addition to relating to its configural properties (Smith 1998), facilitates discrimination between volatiles, such as components of a bouquet that are structurally similar and/or form a substrate-product duo. This seems likely since binary odor mixtures receive a unique representation in the honey bee brain, quite different from its components when viewed separately (Joerges et al., 1997).

Evolutionary development of floral “scent genes” has facilitated the production of novel floral odors. For example, in Clarkia brewri, S-Linalool is produced in a one step reaction catalyzed by S-Linalool Synthase from its ubiquitous precursor, geranyl pyrophosphate (GPP). Its appearance in the bouquet clearly defines it from a closely related species, Clarkia Concinna (Pichersky et al., 1995). The production of Benzyl acetate from Benzyl alcohol by the action of acetyl CoA:benzyl alcohol acetyltransferase (Dudareva et al., 1998), makes benzyl acetate a major constitute of the Clarkia Brewri bouquet. Linalool and Benzyl acetate are some of the most common odor components in flowers, yet in many instances benzyl acetate co-occurs in the volatile emission of the flowers together with its substrate- benzyl alcohol (Knudsen et al., 1993). Since they bear some structural similarities, the bees should have a capability of distinguishing between their presence in the bouquet. When learning particular bouquet components associated with high reward, the bees may use a “blocking” (Smith and Cobey 1994, Hosler and Smith 2000) strategy to relate only to these odors, while ignoring other bouquet constituents.

Learning theory predicts that when two separate excitory (positive) differentially rewarding stimuli are presented in tandem they will elicit a differential acquisition curve (Rescola and Wagner, 1972). Thus, in differential PER conditioning, the response curve to the CS for the more rewarding US will reach a higher asymptote than the lesser rewarded CS. This has clear relevance to decision making in foraging honey bees that are rarely confronted with an all or nothing reward ensemble. This is also the basis of the preference by honeybees of a certain genotype/cultivar in agronomic situations, whereby cross-pollination is required between said genotypes to facilitate, for example, the production of hybrid seed.

U.S. Pat. No. 5,849,526, describes methods for stable transformation of plants with monoterpene synthases, and especially linalool synthase, to, among other things, enhance insect visitation. However, it is known to those of skill in the art that in order for the attractiveness of the target crop to increase, it is necessary to increase the caloric (nectar) or protein (pollen) reward; and further that it is not sufficient to enhance the signal alone for reasons discussed in the aforementioned sections “Attempts to enhance honeybee visitation to flowers” and “The associative learning capabilities of honeybees”.

There is thus a widely recognized need for, and it would be highly advantageous to have, a method that will manipulate the foraging behavior of honeybees in a manner that will decrease their ability to differentiate between two genotypes of same species to facilitate better cross-pollination. An example for such a need is in the case of cross-polination of parental plants in the production of hybrid seeds.

### SUMMARY OF THE INVENTION

According to one aspect of the present invention there is provided a method of enhancing insect assisted cross-pollination between flowering plants of a single plant species, the flowering plants being of at least two different genetic backgrounds (e.g., different cultivars), the method comprising co-expressing in plants of the at least two different genetic backgrounds at least one scent biosynthetic enzyme and growing the plants in a cross-pollination vicinity in a presence of at least one pollinating insect. As used herein the phrase “plant species” refers to all plant genus capable of sexual reproduction.

According to further features in preferred embodiments of the invention described below, plants of the different genetic backgrounds are paternal and maternal lines used for hybrid seed production.

According to still further features in the described preferred embodiments the maternal line is male sterile.

Thus, in a specific embodiment, the present invention provides a method of enhancing insect assisted cross-pollination between parental and maternal lines of plants used in hybrid seed production, the method comprising co-expressing in plants of the parental and maternal lines at least one scent biosynthetic enzyme and growing the plants in a cross-pollination vicinity in a presence of at least one pollinating insect.

According to still further features in the described preferred embodiments-plants of the at least two different genetic backgrounds are characterized by producing differential pollinator rewards.

According to still further features in the described preferred embodiments the differential pollinator rewards include different types of differential pollinator rewards.

According to still further features in the described preferred embodiments the differential pollinator rewards include different amounts of a single differential pollinator reward.

According to still further features in the described preferred embodiments the differential pollinator rewards include different amounts of a single differential pollinator reward and different types of differential pollinator rewards.

According to still further features in the described preferred embodiments plants of the at least two different genetic backgrounds are characterized by producing differential pollinator rewards during at least one given seasonal time period.

According to still further features in the described preferred embodiments the at least one pollinating insect includes bees.

According to still further features in the described preferred embodiments the bees are honeybees.

According to still further features in the described preferred embodiments the bees are bumblebees.

According to still further features in the described preferred embodiments the at least one pollinating insect is selected from the group consisting of a bee, a beetle, a fly and a moth.

According to still further features in the described preferred embodiments the pollinating insect is native to an area in which the plants are grown.

According to still further features in the described preferred embodiments the pollinating insect is man-introduced to an area in-which the plants are grown.

According to still further features in the described preferred embodiments the introduction is via at least one beehive.

According to still further features in the described preferred embodiments the plants are grown in a field.

According to still further features in the described preferred embodiments the plants are grown in a greenhouse.

According to still further features in the described preferred embodiments the plants species is selected from the group consisting of sunflower, cotton, tomato, cucurbits, almond, apple, cherry, pear, kiwi and avocado.

According to still further features in the described preferred embodiments co-expressing the scent biosynthetic enzyme in plants of the different genetic backgrounds is to an extent so as to reduce an ability of the pollinating insect to differentiate between the plants of the different genetic backgrounds.

According to still further features in the described preferred embodiments co-expressing the at least one scent biosynthetic enzyme in plants of the at least two different genetic backgrounds is effected by transforming or infecting the plants with a vector.

According to still further features in the described preferred embodiments the vector is a plant virus.

According to still further features in the described preferred embodiments the plant virus has been modified to restrict a severity of infection symptoms to the plants.

According to still further features in the described preferred embodiments the plant virus has been modified to restrict a natural transfer by an insect-vector.

According to still further features in the described preferred embodiments co-expressing the scent-biosynthetic enzyme in plants of the-at least two different genetic backgrounds is under a control of a constitutive promoter.

According to still further features in the described preferred embodiments co-expressing the at least one scent biosynthetic enzyme in the plants of the at least two different genetic backgrounds is under a control of a tissue specific promoter.

According to still further features in the described preferred embodiments the tissue specific promoter is selected from the group consisting of an epithelial specific promoter, a flower specific promoter and a nectary specific promoter.

According to still further features in the described preferred embodiments the scent biosynthetic enzyme is selected from the group consisting of a monoterpene synthase, an acetyl transferase and a methyltransferase.

According to still further features in the described preferred embodiments the cross-pollination between plants of the at least two different genetic backgrounds is essential and rudimentary.

According to still further features in the described preferred embodiments the cross-pollination between plants of the at least two different genetic backgrounds is beneficial.

According to another aspect of the present invention there is provided a method of overshadowing associative learning of a pollinating insect, the method comprising exposing the pollinating insect to at least two differential pollinator rewards, each of the differential pollinator rewards being scented with an added identical scent. Exposing the pollinating insect to at least two differential pollinator rewards is preferably effected by allowing the pollinating insects to feed on flowering plants of a single plant species, the flowering plants being of different genetic backgrounds and producing the differential pollinator rewards, and the flowering plants are engineered for co-producing at-least one scent biosynthetic enzyme and are therefore scented with the added identical scent.

The present invention successfully addresses the shortcomings of the presently known configurations by providing a novel and advantageous method of enhancing insect assisted cross-pollination between flowering plants.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 a is a schematic presentation of an experimental set up used while reducing the present invention to practice. The experiments were conducted in a screened enclosure (marked by dotted lines) using artificial flowers (marked by circles). The distances (1 m) between the flowers were the same both between and within rows. A syringe pump simultaneously filled either high (20 microliters/flower/minute, 45%) or low (10 microliters/flower/minute, 15%) sucrose solution into either rows 1+3 and 2+4, respectively or to rows 2+4 and 1+3, respectively.

FIG. 1b is a Table demonstrating the experimental setup used while reducing the present invention to practice. The experimental setup is balanced to avoid bias that may be due to positional learning (via changing positions of high and low rewarding flowers from day to day), odour bias (by daily changing the hive used and by using a pseudorandom order of consequent odour presentations) and physical conditions such as temperature and irradiance kept almost constant (via performing the experiments within a 3 week period in the early summer).

FIGS. 2-5 are graphs demonstrating a comparison between the relative mean visitation to the high rewarding artificial flowers between experiments conducted for different combinations of odors. Count stages 1-4=visits+flow of sucrose solution. Count stages 5-6=Visits after cessation of sucrose solution flow (see Examples section for further details). FIG. 2: High rewarding (diamonds)=linalool; Low rewarding (squares)=1-hexanol. FIG. 3: High rewarding (diamonds)=1-hexanol; Low rewarding (squares)=linalool. FIG. 4: High rewarding (diamonds)=linalool+benzyl acetate. Low rewarding (squares)=1-hexanol+benzyl acetate. FIG. 5: High rewarding (diamonds)=1-hexanol+benzyl acetate. Low rewarding (squares)=linalool+benzyl acetate.

### DESCRIPTION OF THE PREFERRED EMBODIMENTS

The present invention is of a method of enhancing entomophilous assisted cross-pollination. Specifically, the present invention is of a method of enhancing entomophilous assisted cross-pollination between flowers of cross-fertilizing genotypes (e.g., cultivars), such as parental genotypes of plants used for the production of hybrid seeds, via co-expression of scent producing enzymes. However, the invention is not limited to monodirectional pollination protocols, rather, it applies also to bidirectional pollination as in the case of two cultivars which serve as pollenizers of one another, so as to enhance fruit production.

The principles and operation of a method according to the present invention may be better understood with reference to the examples and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

According to one aspect of the present invention there is provided a method of enhancing insect assisted cross-pollination between flowering plants of a single plant species. The flowering plants are of at least two different genetic backgrounds, e.g., different cultivars.

As used herein, the phrase “cross-pollination” refers to transfer of pollen from staminate flower parts of a flower of a plant to the pistilate flower parts of another flower on a different plant of the same plant species but of a different genetic background (e.g., cultivar), the plants having non-identical genotypes. For some plant species, cross-pollination between genetic backgrounds is essential and rudimentary. Examples include avocadoes, blueberries, certain apple cultivars and sweet cherry. For other plant species, cross-pollination between different genetic backgrounds is beneficial. Examples include almonds, alfalfa, and many Rosaceae. Additional examples of plants in which cross-pollination is either obligatory or beneficial are well known to the skilled artisan.

Typically, plants of different genetic backgrounds (e.g., cultivars) offer pollinators with differential pollinator rewards.

As used herein, the phrase “differential pollinator reward” refers to a non-equal production at any given time of nectar or pollen by two genotypes (cultivars) of the same plant species. Thus, the differential pollinator rewards can be different amounts of pollinator reward(s) and/or different types of pollinator rewards produced during at least one given seasonal time period.

Associative learning by the pollinating insect, associating the reward with, for example, a scent or scents unique to each of the genotypes (e.g., cultivars), results in frequent visitations to flowers offering the higher reward and less frequent or no visitations to flowers offering the lower reward, thereby cross-pollination is reduced or hampered altogether. This problem is specifically emphasized with respect to parental lines seeded or planted in alternating rows used in the production of hybrid seeds, wherein, in many cases, flowers of the maternal line which is male sterile may produce nectar yet in many cases are designed not to produce pollen, to produce fewer pollen or to produce aberrant, less pollinator rewarding, pollen, whereas flowers of the paternal line produce both nectar and viable pollen.

This problem is reduced or eliminated in accordance with the teachings of the present invention and cross-pollination is enhanced by co-expressing in plants of the at least two different genetic backgrounds (e.g., cultivars) at least one scent biosynthetic enzyme and further by growing the plants in a cross-pollination vicinity in a presence of at least one pollinating insect. The at least one scent biosynthetic enzyme releases in plants of both genetic backgrounds volatiles serving as a masking scent, thereby overshadowing the associative learning process, which results in increase in cross-pollination. Thus, according to preferred embodiments of the invention, co-expressing the scent biosynthetic enzyme in the plants of the different genetic backgrounds is to an extent so as to reduce the ability of the pollinating insect to differentiate between the cultivars.

As used herein, the phrase “pollinating insect” refers to any insect, such as, but not limited to, a bee, a beetle, a fly or a moth that has the capacity of transferring pollen from staminate-flower parts of a flower to the pistilate flower parts of a flower of either the same flower or of another flower, whether on the same plant or on another plant of the same plant species.

As used herein, the phrase “cross-pollination vicinity” refers to a vicinity that allows visitations of flowers of different plants by an individual pollinating insect. As land is a valuable resource, plants grown using commercial agricultural techniques, either in the field or in the greenhouse are seeded or planted in cross-pollination vicinity.

As used herein, the phrase “scent biosynthetic enzyme” refers to an enzyme that catalyzes the conversion of a substrate precursor molecule present in a budding or blooming flower to a volatile product molecule, which when produced volatilizes to the surrounding environment. Examples of scent biosynthetic enzymes include, but are not limited to, monoterpene synthases, acetyl transferases and methyltransferases.

As used herein the phrase “scent biosynthetic gene” refers to a gene encoding a scent biosynthetic enzyme as herein defined. There are a plurality of known cloned scent biosynthetic genes. For example monoterpene synthases have been described in U.S. Pat. No. 5,849,526, which discloses the nucleotide sequence of the enzyme linalool synthase from Clarkia brewri that produces linalool from geranyl pyrophosphate (GPP) in a one step reaction. cDNAs from other monoterpene synthases have been described in U.S. Pat. No. 5,891,697 encoding, for example, 1,8-cineole synthase and (+)-sabinene synthase from common sage (Sa/via officinalis). Limonene synthase's nucleotide sequence is disclosed in U.S. Pat. No. 5,871,988. Other scent biosynthetic enzyme clones have been described in, for example, Dudareva et al. 1998 (Benzyl alcohol:acetyl CoA acetyltransferase, BEAT), Wang and Pichersky 1998 (S-adenosyl-L-methionine: (iso)eugenol O-methyltransferase, IEMT), Ross et al. 1999 (S-Adenosyl-L-Methionine:Salicylic Acid Methyl Transferase S-AMT) and Murfitt et-al. 2000 (S-Adenosyl-L-methionine:benzoic acid carboxyl methyltransferase (BAMT). All aforementioned references to scent biosynthetic genes are incorporated herein in their entirety. SEQ ID NOs: 1, 3, 5, 7, 9, 11 and 13 provide cDNA sequences of genes encoding Linalool synthase (LIS), Limonene synthase, Sabinene synthase (SAS) Acetyl CoA:benzyl alcohol acetyltransferase (BEAT), S-Adenosyl-L-Methionine:Salicylic Acid Methyl Transferase (SAMT), S-adenosyl-L-methionine: (iso)eugenol O-methyltransferase, EMT and S-Adenosyl-L-methionine:benzoic acid carboxyl methyltransferase (BAMT respectivly, whereas SEQ ID NOs:2, 4, 6, 8, 10, 12 and 14 provide the corresponding amino acid sequences.

Based on the sequence information provided herein one can use gene-screening protocols to isolate homologs. Thus, genomic and cDNA libraries can be screened with probes derived from or which are similar to the above sequences or portions thereof. Similarly, databases, such as EST databases can be electronically screened for homologs. Techniques as described in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Oligonucleotide Synthesis” Gait, M. J., ed. (1984); “Nucleic Acid Hybridization” Hames, B. D., and Higgins S. J., eds. (1985); “A Practical Guide to Molecular Cloning” Perbal, B., (1984); “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein, can be used to clone such homologs.

As used herein the term “homologs” refer to resemblance between compared polypeptide or polynucleotide sequences as determined from the identity (match) and similarity (amino acids of the same group) between amino acids that comprise polypeptide sequences or the identity between nucleotides that comprise polynucleotide sequences. Typically homologs share at least 50% sequence similarity. Homolog genes typically share a common ancestral gene.

As used herein, the terms “volatile” and “volatiles” refer to chemicals that are produced in flowers by the action of scent biosynthetic enzymes and are dissipated into the surroundings.

In a specific embodiment, the present invention provides a method of enhancing insect assisted cross-pollination between parental and maternal lines of plants used in hybrid seed production. This method is effected by co-expressing in plants of the parental and maternal lines at least one scent biosynthetic enzyme and growing the plants in a cross-pollination vicinity in a presence of at least one pollinating insect.

Any pollinating insect can be used to implement the method of the present invention provided it evolved during evolution to have associative learning capabilities. As is further described in the Background section above, bees have associative learning capabilities. Since associative learning is an individual characteristic, also other pollinating insects evolved having such capabilities, including, but not limited to, beetles, flies and moths. For the same reasons bees became the preferred pollinators in conventional agriculture, bees are the preferred insect pollinator also according to the present invention. These reasons include not only the effectiveness by which bees cross-pollinate, rather also the ease by which bees can be propagated, handled, shuttled, etc., as most bees congregate in hives, including artificial hives.

Two bees species are most commonly used for agricultural pollination. The first species is the honeybee (Apis mellifera). Honeybees are traditionally used in agriculture to facilitate pollination of plants with a vertical slit along the length of the stamen. However, honeybees are inadequate for pollinating plant species that produce pollen in small smooth grains, which are released from the apical aperture/slit only when the blossom of the plant is shaken. This is due to the inability of the honeybees to shake the blossom in order to release pollen, an insect behavior referred to as “buzz pollination”. Among the species of bees capable of buzz pollination are the bumblebees (Bombus terrestris and other Bombus spp.). The use of bees capable of buzz pollination is known to greatly increase pollination percentage in vegetable crops including tomato, eggplant and other plant species of the Solanum genus, and also improves the quality of the vegetables by increasing the number of pollinated seeds per blossom.

According to the present invention, the pollinating insect can be native to the area in which the plants are grown or it can be man-introduced to that area, by for example, placing beehives, or by spreading a non-congregating insect species.

The method of enhancing cross-pollination in accordance with the teachings of the present invention is useful for both field and greenhouse crops. Plants which can be cross-pollinated using the method of the present invention include, but are not limited to, tomato, artichoke, cucurbits (watermelon, melon, cucumbers etc.), onion, sunflower, cotton, alfalfa clover and many other plants.

Co-expressing the scent biosynthetic enzyme(s) in the plants is effected according to the present invention using transformation or infection with suitable vectors.

A construct according to the present invention includes a scent biosynthesis gene (e.g., either cDNA, genomic DNA or composite DNA including both genomic and cDNA derived sequences) operably linked downstream of a plant promoter which directs its expression.

As used herein, the phrase “complementary DNA” includes sequences that originally result from reverse transcription of messenger RNA using a reverse transcriptase or any other RNA dependent DNA polymerase. Such sequences can be subsequently amplified in vivo or in vitro using a DNA dependent DNA polymerase.

As used herein, the phrase “genomic DNA” includes sequences that originally derive from a chromosome and reflect a contiguous portion of a chromosome.

As used herein, the phrase “composite DNA” includes sequences which are at least partially complementary and at least partially genomic.

Numerous plant functional expression promoters and enhancers which can be either tissue specific, developmentally specific, constitutive or inducible can be utilized by constructs of the present invention, some examples are provided hereinunder.

As used herein the phrase “plant promoter” or “promoter” includes a promoter which can direct gene expression in plant cells (including DNA containing organelles). Such a promoter can be derived from a plant, bacterial, viral, fungal or animal origin. Such a promoter can be constitutive, i.e., capable of directing high level of gene expression in a plurality of plant tissues, tissue specific, i.e., capable of directing gene expression in a particular plant tissue or tissues, inducible, i.e., capable of directing gene expression under a stimulus, or chimeric, i.e., formed of portions of at least two different promoters.

Examples of constitutive plant-promoters include, without being limited to, CaMV35S and CaMV19S promoters, FMV34S promoter, sugarcane bacilliform badnavirus promoter, CsVMV promoter, Arabidopsis ACT2/ACT8 actin promoter, Arabidopsis ubiquitin UBQ 1 promoter, barley leaf thionin BTH6 promoter, and rice actin promoter.

Examples of tissue specific promoters include, without being limited to, bean phaseolin storage protein promoter, DLEC promoter, PHSβ promoter, zein storage protein promoter, conglutin gamma promoter from soybean, AT2S gene promoter, ACTI 1 actin promoter from Arabidopsis, napA promoter from Brassica napus and potato patatin gene promoter.

The inducible promoter is a promoter induced by a specific stimuli such as stress conditions comprising, for example, light, temperature, chemicals, drought, high salinity, osmotic shock, oxidant conditions or in case of pathogenicity and include, without being limited to, the light-inducible promoter derived from the pea rbcS gene, the promoter from the alfalfa rbcS gene, the promoters DRE, MYC and MYB active in drought; the promoters INT, INPS, prxEa, Ha hsp 17.7G4 and RD21 active in high salinity and osmotic stress, and the promoters hsr2O3J and str246C active in pathogenic stress.

In context of the present invention, it is advantageous that catalysis of volatiles will predominant in flowers. Thus, if the catalyzed substrate is unique to, or more overly abundant in, flowers relative to other plant tissues, a constitutive promoter can be employed, the expression through which results in volatiles released most particularly from the flowers. If, on the other hand, the substrate is present in the flower as well as other plant tissues to a similar extent, then a flower specific promoter is preferably employed, again the expression through which results in volatiles released from the flowers only.

As used herein the phrase “flower specific promoter” refers to a promoter that is active in a flower tissue, such as, but not limited to, chsA (chalcone synthase) from Petunia hybrida or other flower specific promoters as were identified specifically for scent biosynthetic enzymes, such as the Linalool Synthase (LIS) promoter from Clarkia brewri.

Alternatively, a nectary specific promoter such as the NEC1 promoter from Petunia hybrida (Ge et al., 2000) can be used.

A construct according to the present invention preferably further includes an appropriate and unique selectable marker, such as, for example, an antibiotic resistance gene. In a more preferred embodiment according to the present invention the construct further includes an origin of replication.

A construct according to the present invention is preferably a shuttle vector, which can propagate both in E. coli (wherein the construct comprises an appropriate selectable marker and origin of replication) and be compatible for propagation in plant cells, or integration in the genome, of a plant. A construct according to the present invention can be, for example, a plasmid, a bacmid, a phagemid, a cosmid, a phage, a virus or an artificial chromosome.

Thus, a nucleic acid construct used according to the method of the present invention is utilized to express in either a transient or a stable manner a structural gene contained therein within a whole plant, defined plant tissues, or defined plant cells.

There are various methods of introducing nucleic acid constructs into both monocotyledonous and dicotyledenous plants (Potrykus, I., Annu. Rev. Plant. Physiol., Plant. Mol. Biol. (1991) 42:205-225; Shimamoto et al., Nature (1989) 338:274-276). Such methods rely on either stable integration of the nucleic acid construct or a portion thereof into the genome of the plant, or on transient expression of the nucleic acid construct in which case these sequences are not inherited by a progeny of the plant.

In addition, several methods exist in which a nucleic acid construct can be directly introduced into the DNA of a DNA containing organelle suchasa-chloroplast.

There are two principle methods of effecting stable genomic integration of exogenous sequences such as those included within the nucleic acid constructs of the present invention into plant genomes:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al., Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:2Q6-209; by the use of micropipette systems: Neuhaus et al., Theor.

Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration.

The Agrobacterium system is especially viable in the creation of transgenic dicotyledenous plants.

There are various methods of direct DNA transfer into plant cells. In electroporation, protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals, tungsten particles or gold particles, and the microprojectiles are physically accelerated into cells or plant tissues.

Following transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Transient expression methods which can be utilized for transiently expressing the isolated nucleic acid included within the nucleic acid construct of the present invention include, but are not limited to, microinjection and bombardment as described above but under conditions which favor transient expression, and viral mediated expression wherein a packaged or unpackaged recombinant virus vector including the nucleic acid construct is utilized to infect plant tissues or cells such that a propagating recombinant virus established therein expresses the non-viral nucleic acid sequence.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al., Science (1986) 231:1294-1297; and Takamatsu et al. FEBS Letters (1990) 269:73-76.

When the virus is a DNA virus, the constructions can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of the present invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

Thus, there are many methods known to those skilled in the art for introducing foreign genes into plants. One particular method, which is described in, for example, Toth et al. 2001 (and references cited therein, especially Dolja et al. 1992), Arazi et al. 2001 and/or in U.S. Pat. No. 5,618,699, provide further insight to the use of plant viruses as vectors for transient gene expression, and are incorporated herein by reference. A cloned DNA fragment is introduced into the virus by either Polymerase Chain Reaction (PCR) cloning, ligation of a restriction fragment or by other methods known to those of skills. In one particular embodiment, a nucleotide sequence encoding Benzyl alcohol:acetyl CoA acetyltransferase (BEAT) (Dudareva et al 1998b) is amplified by PCR with introduction of specific restriction enzyme recognition sequences in the primers of the amplification reaction, said restriction enzyme recognition sequences corresponding to similar sequences found on a recombinant plasmid clone of Zucchini Yellow Mosaic Virus (ZYMV) (Gal-On et al., 1992) at a specific site of insertion, in a manner that places the BEAT upstream of the coat protein sequence, but with an added protease recognition sequence to facilitate disunion of the polypeptide. After ligation, electroporation into E. coli, amplification and purification of the recombinant plasmid DNA by methods known to the skilled artisan, the DNA can be introduced into genotypes of all Cucurbitaceae species via, for example, particle bombardment (Gal-On et al., 1995). In one particular embodiment these Cucurbitacea are cultivars (different genotypes of the same species) used to produce hybrid seed, planted in the field to facilitate cross-pollination in ways known to those of skill. Subsequent multiplication of viral RNA from introduced recombinant DNA causes high expression of BEAT, and its interaction with a benzyl alcohol substrate produces benzyl acetate. In the flower and other epithelial plant tissues, said benzyl acetate volatilizes. Simultaneous appearance of benzyl acetate in these two cultivars reduces the ability of bees to discriminate between the cultivars and thus increase cross-pollination and yield of hybrid seed.

According to preferred embodiments of the present invention, the plant virus that is used for infection is a modified virus so as to restrict a severity of infection symptoms to the infected plants.

Some potyvirus vectors have already been developed (e.g., TEV, Dolja, 1998, ZYMV, Gal-On et al., 1992, Arazi et al. 2001), and there is a lot of data regarding their cloning and characteristics. One determinant for severity is also known. The single mutation FRNK (SEQ ID NO:15) to FINK (SEQ ID NO:16) in the helper component viral protein (HC) confers mildness of the symptom of ZYMV without affecting the replication (Gal-On and Raccah, 2000). Therefore it can be introduced to infectious potyvirus clones by directed mutagenesis in order to engineer attenuated clones.

Determinants for aphid transmission are also known. One mutation in the coat protein (CP) (namely DAG (SEQ ID NO:17) to DTG (SEQ ID NO: 18), Atreya et al., 1990, Gal-On et al., 1992), and two in the HC (KLSC (SEQ ID NO:19) to (SEQ ID NO:20) ELSC Atreya et al., 1992 or PTK (SEQ ID NO:21) to PAK (SEQ ID NO:22), Huet et al., 1994) abolish the transmission. Thus, it is possible to design a potyvirus mutant which contains all these 3 mutations, and which will be absolutely aphid non transmissible and attenuated.

A technique for introducing exogenous nucleic acid sequences to the genome of the chloroplasts or chromoplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

Gene knock-in can also be used to transform a plant to express an exogene according to the present invention, by positioning such a gene on a chromosome downstream of a functional promoter. A knock-in construct typically includes positive and negative selection markers and may therefore be employed for selecting for homologous recombination events. One ordinarily skilled in the art can readily design a knock-in construct including both positive and negative selection genes for efficiently selecting transformed plant cells that underwent a homologous recombination event with the construct. Such cells can then be grown into full plants. Standard methods known in the art can be used for implementing a knock-in procedure. Such methods are set forth in, for example, U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, Methods in Enzymology, 194:251-270, 1991; Capecchi, Science 244:1288-1292, 1989; Davies et al., Nucleic Acids Research, 20 (11) 2693-2698, 1992; Dickinson et al., Human Molecular Genetics, 2(8):1299-1302, 1993; Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991; Jakobovits et al., Nature, 362:255-261 1993; Lamb et al., Nature Genetics, 5: 22-29, 1993; Pearson and Choi, Proc. Natl. Acad. Sci. USA, 1993, 90:10578-82; Rothstein, Methods in Enzymology, 194:281-301, 1991; Schedl et al., Nature, 362: 258-261, 1993; Strauss et al., Science, 259:1904-1907, 1993, WO 94/23049, WO93/14200, WO 94/06908 and WO 94/28123 also provide information.

According to another aspect of the present invention there is provided a method of overshadowing-associative learning of a pollinating insect. This method is effected by exposing the pollinating insect to at least two differential pollinator rewards, each of the at least two differential pollinator rewards being scented with an added identical scent. Exposing the pollinating insect to at least two differential pollinator rewards is preferably effected by allowing the pollinating insects to feed on flowering plants of a single plant species, the flowering plants producing the at least two differential pollinator rewards, and the flowering plants co-producing at least one scent biosynthetic enzyme and are therefore scented with the added identical scent.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following example.

### EXAMPLES

Reference is now made to the following Example 1, which together with the above descriptions, illustrate the invention in a non-limiting fashion.

### EXAMPLE 1

Manipulating honeybees foraging behavior via adding benzyl acetate to visually identical artificial flowers that are secreting differential sucrose reward and are associated with differential odors MA TERIALS, EXPERIMENTAL SET-UP AND METHODS

Four honeybee colonies of the Buckfast line were used, kept in 10-comb standard Langstroth hives. They were concurrently introduced into a 12×7 meter screened enclosure in Rehovot, Israel, and remained there throughout the duration of the experiment held during May. In order to sustain the colonies, and to maintain a constant motivation for foraging, they were fed twice a week with 0.5 L of 100% (w/v) sugar syrup and a 100 grams pollen supplement patty.

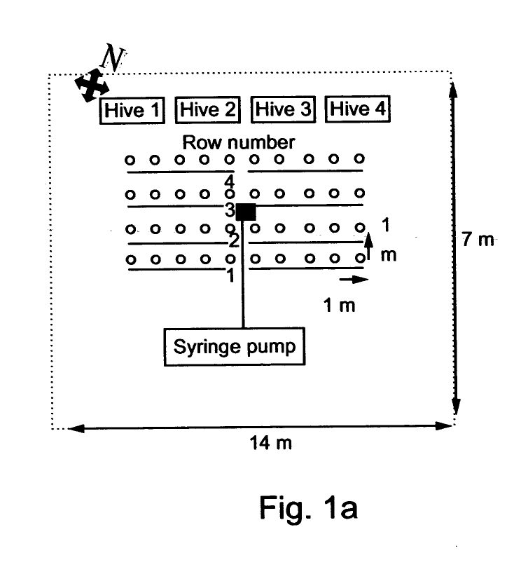

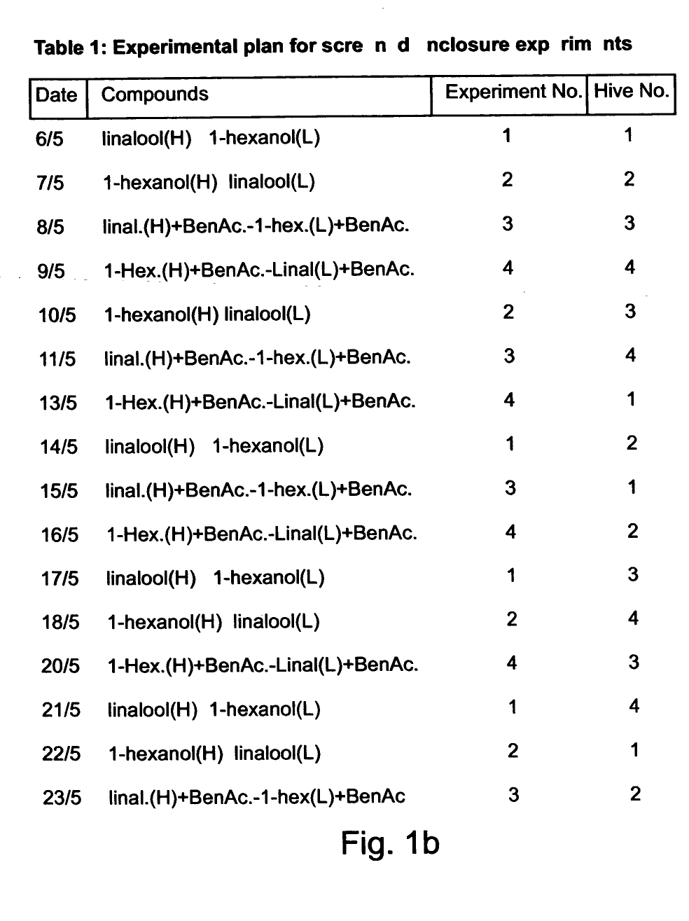

During an experiment, bees foraged from a patch of 40 artificial flowers, distributed along four rows (like crops in agricultural fields), with 1 m separating between rows and between flowers (FIG. 1). The ten flowers of each of two lines offered a high reward and the ten flowers of each of the other two lines offered a low reward (see experiment description for details).

Artificial Flowers

Flowers were constructed using Plexiglas. A 10-mm thick, 6 cm in diameter, piece constituted a flower, and was mounted on a 3-mm thick, 14.5×14.5 cm, green base. At the center of each flower, an 8.5-mm deep well, 5 mm in diameter, was made, which could hold about 100 μl of sugar solution. Tubing reached the well by a tunnel drilled through the flower.

Flowers were covered by yellow circular labels with four blue strips acting as nectar guides, pointing towards the center. The well at the center was marked by a blue circle 2 cm in diameter. Two strips of filter paper, 20×5 mm each, were glued on either side of the feeding well, with the odorants (3 μl per strip) administered onto the strips with a calibrated pipettor. Thus, all the flowers looked the same, but the flowers in the High and the Low rewarding lines were distinguished by the odorants applied to them, according to the specifications of each experiment.

General Procedure

Experiments began 30-90 minutes after first light, as soon as the ambient temperature reached about 19° C. Each trial lasted for approximately 70 minutes, with the ambient temperature at the end of each trial reaching about 25° C. This ensured that evaporation of both sugar solution and odors was moderate and almost constant throughout the experimental period. The entrances of the hives were blocked approximately half an hour before first light, except for the hive that participated in the experiment on that day. At the beginning of every trial one researcher applied the odorants (2×3 microlitres/flower) with a hand-held pipette, while the other operated the automatic syringe pump to start the flow of sucrose solution into the flowers.

To assess the bees' ability to discriminate between High and Low rewarding flowers, each row was assigned a position (1-4). A researcher moved from flower to flower along each row and counted for 10 seconds the number of bees that touched the inner blue circle (“pollination event”) of each flower. Each round of counting the bees on all 40 flowers (˜10 minutes) constituted a count episode. Each day one replicate was performed of every experiment, during which six count episodes were conducted consecutively, with a short break between the third and fourth count episodes when a second round of odorant application was performed to compensate for evaporation. The syringe pump was turned off after the fourth count episode, and count episodes 5 and 6 became extinction episodes. Four replicates of each experiment were performed. Although these replicates were performed on consecutive days, experiments and hives were alternated to avoid learning of combinations from day to day (see Table in FIG. 1b). In addition, the position of the High and Low rewarding rows was altered between days to control for position effects such as positional learning. After each replicate, the entrances of all the hives in the enclosure were opened and the bees were allowed to scout the non-rewarding artificial flowers.

In all experiments four syringes (2×50 ml and 2×20 ml) were mounted on an automated syringe pump (SP 200, World Precision Instruments Inc., Sarasota, USA) and delivered sucrose solution into the flowers. The 50 ml syringes were filled with a 45-% w/v sucrose solution (line H, High reward) and the 20 ml syringes were filled with a 15% w/v sucrose solution (line L, Low reward). The flow rates were 0.2 ml/min and 0.1 ml/min for the H and L lines, respectively. This amounted to a total flow of 20 μl/flower/minute for the H line and 10 μl/flower/minute for the L line. Thus, the H line received six times the total sugar reward of line “B”. Each syringe was connected to two pieces of a 6-m long, 1.6 mm internal diameter Tygon tubing (Fisher Scientific Company, Pittsburgh, USA). The tubing was spread in four parallel rows, alternating between H and L lines. Artificial flowers were connected to the main lines with 20-cm long, 0.8 mm ID tubing and an infusion tap that controlled flow into each flower.

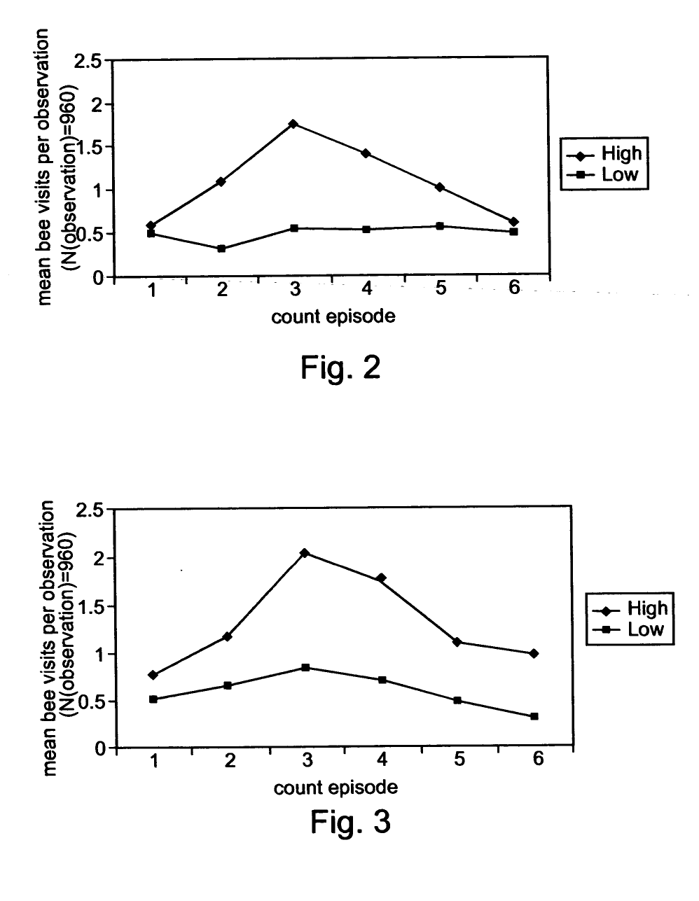

Experiments 1 and 2 (Table 1 in FIG. 1b, FIGS. 2 and 3) Ability to Associatively Learn the Position of the High Rewarding Flowers

To find whether the bees could learn to prefer the high rewarding flowers via associating a given odor with it, either linalool or 1-hexanol were applied to the high rewarding flowers, and the same odors reciprocally to the low rewarding flowers. This also permitted to establish if the bees have an innate preference to either linalool or 1-hexanol, since their appearance in natural bouquets is disproportionately in favor of linalool (Knudsen et al 1993). These experiments reflect the ability of the bees in their natural settings to detect predictive differences in reward salience via using standard associative “measures”.

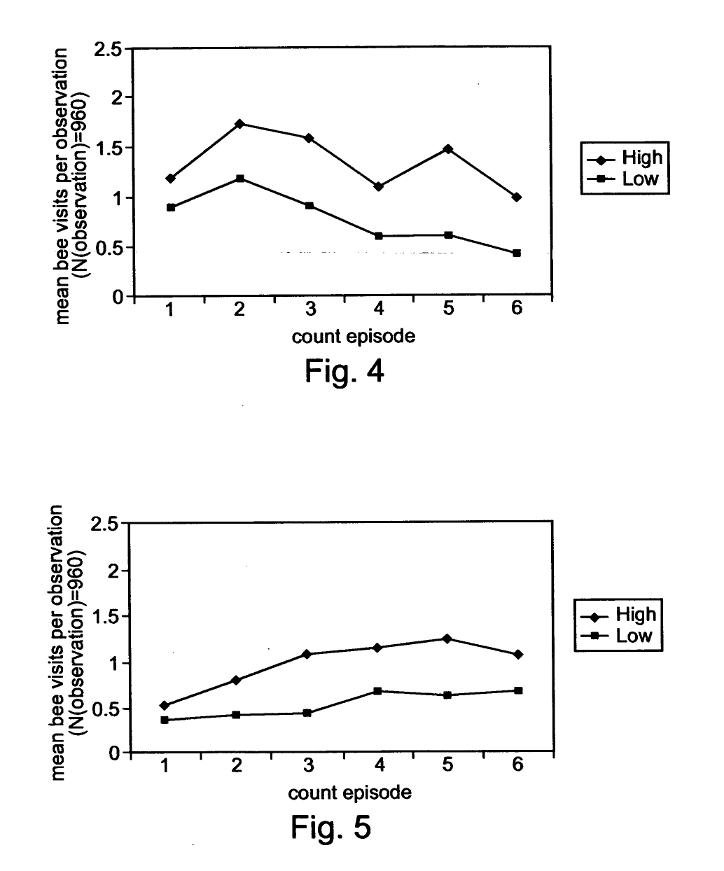

Experiments 3 and 4 (Table 1 in FIG. 1b, FIGS. 4 and 5) Effect of Adding an Additional Odor (Benzyl Acetate) to the Combinations of Experiments 1 and 2

The ability to reduce the bees' ability to differentiate between the High and Low rewarding odors by adding a supplemental odor (benzyl acetate) was examined. This was done by concurrently introducing benzyl acetate to both High and Low rewarding flowers together with linalool and 1-Hexanol, when these are alternately associated with the High and Low rewards. The dispensing of the sucrose solution remained identical to experiments 1 and 2. These experiments reflect the ability or inability of the bees to overcome reduced predictive differences of reward, as exemplified by the presence of a major common odorant

### Experimental Results

FIGS. 2-5 demonstrate that the statistic used, i.e., Mean Bee visits per Flower per Observation (mBeeFO), was successful in identifying the differential bee visitation (Δ) between High and Low rewarding flowers. Moreover, the value ΔmBeeFO, was useful in distinguishing the capacity of the added odor, benzyl acetate, in overshadowing the ability of the bees to learn the identity of the more highly rewarding flowers. The ΔmBeeFO value was almost identical at the beginning of each experiment in each day. However, ΔmBeeFO at count stage 3, for example, for experiments where linalool and 1-hexanol were used as High and Low rewarding associated odors, reciprocally, were 1.5 and 1.4 respectively. When benzyl acetate was added as the overshadowing odor there were either more visits to the lower rewarding flowers by count stage 3 (when the higher rewarding flowers were associated with linalool) or less visits to the higher rewarded flowers (when these were associated with 1-hexanol). Most importantly, the value of ΔmBeeFO at count stage 3 was reduced to 0.65 and 0.6 for linalool and 1-hexanol associated flowers, respectively.

Interestingly, at count episode 4, there was a reduction of ΔmBeeFO in experiments 1 and 2. However, this could be due to a saturation of bees that resulted in a reduction of the actual reward that every bee was confronted with, subsequently leading to extinction learning. This will probably not be the situation in a agricultural field situation, where saturation is less likely. Thereafter count episodes 5 and 6 only reinforced the extinction effect.

Thus, the common odorant benzyl acetate, masks/overshadows and “confuses” the bees, and differentiation (ΔmBeeFO) is significantly reduced compared to when only one different structurally unrelated compound is associated with the differential reward.

Practically, honeybee acquired recognition of a more rewarding cultivar often hampers successful cross-pollination (Pham-Delegue et al., 1989). Since the value of honeybees to pollination of modern crops is enormous (Robinson et al 1989), reducing the differentiating capacity of the bees using introduced co-occurring odors according to the teaching of the present invention, may facilitate better cross-pollination.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such-reference is available as prior art to the present invention.

### REFERENCES CITED

(Additional References are Cited in the Text)

Ambrose J. T., Shulthesis J. R., Bambara S. B., Mangum W. (1995). An evaluation of selected commercial bee attractants in the pollination of cucumbers and watermelons. Am. Bee J. 135:267-271.

Arazi T., Slutsky S. G., Shiboleth Y. M., Wang Y., Rubinstein M., Barak S., Yang J., Gal-On A. (2001). Engineering zucchini yellow mosaic potyvirus as a non-pathogenic vector for expression of heterologous proteins in cucurbits. J Biotechnol. 87(1):67-82.

Atreya, C. D., Raccah, B. and Pirone, T. P. (1990). A point mutation in the coat protein abolishes aphid transmissibility of a potyvirus. Virology 178, 161-165.

Baker H. G., Baker I.(1977). Non sugar chemical constituents of nectar. Apidologie. 8:349-356. Baker H. G., Baker I. (1983). Floral nectar sugar constituents in relation to pollinator type. In: Handbook of experimental pollination biology. (C. E. Jones and R. J. Little, eds.), Scientific and academic editions, Van Nostrand Reinhold Co., New York, N.Y. pp.117-141.

Bestmann H. J., Winkler L., von Helversen O. (1997) Headspace analysis of volatile flower scent constituents of bat-pollinated plants. Phytochemistry 46(7):1169-1172.

Bhagavan S, Smith B H (1997) Olfactory conditioning in the honey bee, Apis mellifera: effects of odor intesity. Physiol Behav 61(1):107-17.

Blight M. M., Le Metayer M., Pham-Delegue M. H., Pickett J. A., Marion-Poll F., Wadhams L. J. (1997) Identification of floral volatiles involved in recognition of oilseed rape flowers, Brassica napus by honeybees Apis mellifera. J. chem. Ecol., 23 1715-1727.

Borg-Karlson A. K., Englund F. O., Unelius C. R. (1994a). Dimethyl oligosulphides, major volatiles released from Sauromatum guttatum and Phallus impudicus. Phytochemistry 35:321-323.

Borg-Karlson A. K., Valterova I., Nilsson L. A. (1994b). Volatile compounds from flowers of six different species in the family Apiaceae: Bouquets for different pollinators? Phytochemistry. 35:111-119.

Currie R., Winston M. L., Slessor K. N., Mayer D. F. (1992a). Effect of synthetic queen mandibular pheromone sprays on fruit crops by honeybees (Hymenoptera: Apidae). J. Econ. Entomol. 85:1293-1299.

Currie R., Winston M. L., Slessor K. N., Mayer D. F. (1992b). Effect of synthetic queen mandibular pheromone sprays on honeybee (Hymenoptera: Apidae) pollination of berry crops. J. Econ. Entomol. 85:1300-1306.

De-Hazan M., Lensky Y., Cassier P. (1989). Effects of queen honeybee (Apis mellifera L.) ageing on her attractiveness to workers. Comp. Biochem. Physiol. 93:777-783.

Dobson H. E. M., Bergstrom J., Bergstrom G., Groth I. (1987). Pollen and flower volatiles in two Rosa species. Phytochemistry 26:3171-3173.

Dobson H. E. M., Groth I., Bergtrom G. (1996). Pollen advertisement: Chemical contrasts between whole-flower and pollen odors. Am. J. Bot. 83:877-885.

V V Dolja, H J McBride, and J C Carrington (1992) Tagging of Plant Potyvirus Replication and Movement by Insertion of β-Glucuronidase into the Viral Polyprotein PNAS 89: 10208-10212.

Dudareva N, Cseke L, Blanc VM, Pichersky E. (1996) Evolution of floral scent in Clarkia: novel patterns of S-linalool synthase gene expression in the C. breweri flower. Plant Cell. 8(7):1137-48

Dudareva N, Raguso R A, Wang J, Ross J R, Pichersky E (1998a) Floral scent production in Clarkia breweri. III. Enzymatic synthesis and emission of benzenoid esters Plant Physiol. 116(2):599-604.

Dudareva N, D'Auria J. C., Nam K. H., Raguso R. A. Pichersky E (1998b) Acetyl-CoA:benzylalcohol acetyltransferase—an enzyme involved in floral scent production in Clarkia breweri. Plant J 14(3):297-304.

Dudareva N., Piechulla B., Pichersky E (1999) Biogenesis of floral scents. Hort. Rev. 24:31-54.

Elmstrom G. W., Maynard D. N. (1990). Attraction of Honey bees to watermelon with bee-attractant. Proc. Fla. State Hort. Soc. 103:130-133.

Faber T., Joerges J., Menzel R. (1999). Associative learning modifies neural representations of odors in the insect brain. Nat. Neurou. 2:74-78.

Fiala A., Muller U., Menzel R. (1999) Reversible downregulation of protein kinase A during olfactory learning using antisense technique impairs long-term memory formation in the honeybee, Apis mellifera J Neurosci 19(22):10125-34.

Gal-On A., Antigunus A., Rosner A., Raccah B. (1992). A zucchini yellow mosaic virus coat protein gene mutation restores aphid transmitability but has no effect on multiplication. J. Gen. Virol. 73:2183-2187.

Gal-On A., Meiri E., Huet H., Hua W. J., Raccah B., Gaba V. (1995). Particle bombardment drastically increases the infectivity of the cloned DNA of zucchin yellow mosaic potyvirus. J. Gen. Virol. 76:3223-3227.

Gal-On A, and Raccah B, 2000. A point mutation in the FRNK motif of the potyvirus HC-pro gene alters the symptom expression in cucurbits and exhibits protection against severe homologous virus. Phytopath. 90, 1056.

Ge Y X, Angenent G C, Wittich P E, Peters J, Franken J, Busscher M, Zhang L M, Dahlhaus E, Kater M M, Wullems G J, Creemers-Molenaar T. (2000). NEC1, a novel gene, highly expressed in nectary tissue of Petunia hybrida. Plant J. 24(6):725-34.

Angenent G C, Wittich P E, Peters J, Franken J, Busscher M, Zhang L M, Dahlhaus E, Kater M M, Wullems G J, Creemers-Molenaar T. (2000) NEC1, a novel gene, highly expressed in nectary tissue of Petunia hybrida Plant J 24:725-34

Gerber B, Smith B H (1998) Visual modulation of olfactory learning in honeybees.” J Exp Biol 201:2213-7.

Gerber B., Geberzahn N., Hellstern F., Klein J., Kowalksy O., Wustenberg D., Menzel R. (1996). Honey bees transfer olfactory memories established during flower visits to a proboscis extension paradigm in the laboratory. Animal Behavior, 52: 1079-1085.

Getz W. M., Smith B. H., (1990) Odorant moiety and odor mixture perception in free-flying honeybees (Apis mellifera) Chemical Senses. 15: 111-128.

Hammer M., Menzel R. (1995). Learning and memory in the Honeybee. J. Neurosci. 15:1617-1630

Hansted L., Jackobson H. B., Olsen C. E. (1994). Influence of temperature on the rhythmic emission of volatiles from Ribes nigrum flowers in situ. Plant. Cell. Envirom. 17:1069-1072.

Henning J. A., Peng Y. S., Montague M. A., Teuber L. R. (1992). Honey bee behavioral response to primary Alfalfa floral volatiles. J. Econ. Entomol. 85:233-239.

Hosler J. S. Smith B. H. (2000) Blocking and the detection of odor components in blends. J. Exp. Biol. 203: 2797-2806.

Huet, H., Gal-On, A., Meir, E. Lecoq, H. and Raccah, B. (1994). Mutations in the helper component (HC) gene of zucchini yellow mosaic virus (ZYMV) affect aphid transmissibility. J. Gen. Virol. 75, 1407-1414.

Jakobsen H. B., Olsen C. E. (1994). Influence of climatic factors on emission of flower volatiles in situ. Planta 192:365-371.

Jackobson H. B., Kristjansson K., Rohde B., Terkildsen M., Olsen C. E. (1995). Can social bees be influenced to choose a specific feeding station by adding the scent of the station to the hive air? J. Chem. Eco. 21:1635-1648.

Joerges J., Kuttner A., Galizia G., Menzel R. (1997) Representations of odor mixtures visualized in the honeybee brain. Nature 387:285-288.

Kim Y. S., Smith B. H. (2000) Effect of an amino acid on feeding preferences and learning behavior in the honey bee, Apis mellifera. J. Insect Physiol May 1, 2000;46(5):793-801.

Knudsen J. T., Tollsten L., Bergstrom G. (1993). Floral scents- a checklist of volatile compounds isolated by headspace techniques. Phytochemistry 33:253-280.

Laska M., Galizia C. G., Giurfa M., Menzel R., (1999) Olfactory discrimination ability and odor structure-activity relationships in honeybees. Chem. Senses. 24:429-438.

Le Metayer M, Marion-Poll F, Sandoz J C, Pham-Delegue M H, Blight M M, Wadhams L J, Masson C, Woodcock C M (1997) Effect of conditioning on discrimination of oilseed rape volatiles by the honeybee: use of a combined gas chromatography-proboscis extension behavioral assay. Chem Senses 22(4):391-8.

Loughrin J. H., Hamilton-Kemp T. R., Burton H. R., Andersen R. A., Hildebrand D. F. (1992). Glycosidically bound volatile components of Nicotiana sylvesteris and N. suaveolens flowers. Phytochemistry 31:1537-1540.

Loughrin J. H., Hamilton-Kemp T. R., Burton H. R., Andersen R. A.(1993). Effect of diurnal sampling on the headspace composition of detached Nicotiana suaveolens flowers. Phytochemistry 32:1417 1419.

Masson C., Pham-Delegue M. H., Fonta C., Gascuel J., Arnold G., Nicolas G., Kerszberg M. (1993). Recent advances in the concept of adaptation to natural odor signals in the honeybee, Apis mellifera L. Apidologie 24:169-194.

Menzel R. (1993). Associative learning in honeybees. Apidologie 24:157-168.

Menzel R., Muller U. (1996) Learning and memory in honeybees: from behavior to neural substrates. Ann. Rev. Neurosci. 19:379-404.

Muller D., Gerber B., Hellstern F., Hammer M., Menzel R. (2000) Sensory precondiin honeybees. J Exp Biol 2000 April; 203 Pt 8:1351-64.

Murfitt L. M., Kolosova N., Mann C. J., Dudareva N. (2000) Purification and characterization of S-adenosyl-L-methionine:benzoic acid carboxyl methyltransferase, the enzyme responsible for biosynthesis of the volatile ester methyl benzoate in flowers of Antirrhinum majus. Arch Biochem Biophys 382:145-51.

Nielsen J. K., Jakobsen H. B., Friis P., Hansen K., Moller J., Olsen C. E.(1995). Asynchronous rhythms in the emission of volatiles from Hesperis matrolnalis flowers. Phytochemistry 38:847-851.

O'neill S. D., Naddeau J. A., Zhang Z. S., Bui A. Q., Halevy A. H. (1993). Interorgan regulation of ethylene biosynthesis genes by pollination. Plant Cell 5:419-432.

Page R. E. Jr, Erber J., Fondrk M. K. (1998) The effect of genotype on response thresholds to sucrose and foraging behavior of honey bees (Apis mellifera L.). J Comp Physiol [A] 1998 April; 182(4):489-500.

Pellmyr O., Bergstrom G., Groth I. (1987). Floral fragrances in Actaea, using differential chromatograms to discern between floral and vegtative volatiles. Phytochemistry 26:1603-1606.

Pelz C., Gerber B., Menzel R. (1997). Odorant intensity as a determinant for olfactory conditioning in honeybees: roles in descrimination, overshadowing and memory consolidation. J Exp Biol 200(Pt4):837-47

Pham-Delegue M. H., Etievant P., Guichard E., Masson C. (1989). Sunflower volatiles involved in Honeybee discrimination among genotypes and flowering stages. J. Chem. Ecol. 15:329-343.

Pichersky E., Raguso R. a., Lewinsohn E., Croteau R. (1994). Floral scent production in Clarkia (Onagraceae). J. Chem. Ecol. 16:3053-3065.

Pichersky E., Lewinson E., Croteau R., (1995). Purification and characterization of S-Linaool Synthase, an enzyme involved in the production of floral scent in Clarkia brewri. Archives Biochem. Biophy. 316:803-809.

Rapp R., Lensky Y., Rabinowitch H. D. (1984). An evaluation of Beeline® as a bee attractant to cucumbers and its effect on hybrid seed production. J. Apicult. Res. 23:50-54.

Ray S., Ferneyhough B. (1999) The effects of age on olfactory learning and memory in the honey bee Apis mellifera. Neuroreport Feb. 10, 1997;8(3):789-93.

Rescorla R. A., Wagner A. R. (1972) A theory of Pavlovian conditioning: variations of the effectiveness of reinforcement and nonreinforcement. In Classical Conditioning. II Current Research and theory (ed. A. H. Black and W. T. Prokasy), pp 64-99. New York: Appleton-Century-Crofts.

Robinson W. S., Nowogrodszki R., Morse R. A. (1989). The value of honeybees as pollinators of U.S. crops (I). Am. Bee. J. 129:477-487.

Ross J. R., Hee Nam K., D'Auria J. C., Pichersky E (1999). S-Adenosyl-L-Methionine:Salicylic Acid Methyl Transferase, an enzyme involved in floral scent production and plant defence, represents a new class of plant Methyltransferases. Arch. Biochem. Biophys. 367(1):9-16.

Sandoz J. C., Laloi D., Odoux J. F., Pham-Delegue M. H., (2000). Olfactory information transfer in the honeybee: compared efficiency of classical conditioning and early exposure. Animal Behavior 59:1025-1034.

Scheiner R, Erber J, Page R. E. Jr (1999) Tactile learning and the individual evaluation of the reward in honey bees (Apis mellifera L.). J Comp Physiol [A] 1999 July; 185(1): 1-10 Schiestl F. P., Ayasse M., Hannes F. Paulus A, Lafstedt C., Hansson W. S., Ibarra F., Francke W. (1999). Orchid pollination by sexual swindle Nature 399: 421.

Schultheis J. R., Ambrose J. T. Bambara S. B., Mangum W. A. (1994). Selective bee attractants did not improve Cucumber and Watermelon yield. HortScience. 29:155-158.

Seeley T. D., Levien R. A. (1985). Social foraging by honeybees; How a colony tracks rich sources of nectar. In: Neurobiology and behavior of honeybees. pp. 38-53.

Smith B. H. (1991). The olfactory memory of the honeybee Apis mellifera. J. Exp. Biol. 161:367-382.

Smith B H (1998) Analysis of interaction in binary odorant mixtures. Physiol Behav 65(3):397-407.

Smith B. H., Cobey S. (1994). The olfactory memory of the honeybee Apis mellifera II: Blocking between odorants in binary mixtures. J. Exp. Biol. 195:91-108.

Song J. T., Seo H. S., Lee J. S., Choi Y. D. (2000). NTR1 encodes a floral nectary-specific gene in Brassica campestris L. ssp. Pekinensis. Pl. Mol. Biol. 42:647-655.

Stopfer M., Bhagavan S., Smith B. H., Laurent G. (1997) Impaired odor discrimination on desynchronization of odor-encoding neural assemblies. Nature 390:70-74

Tollsten L., Bergstrom G. (1988). Headspace volatiles of whole plants and macerated plant parts of Brassica and Sinapis. Phytochemistry 27:2073-2077.

Toth R. L., Chapman S., Carr F., Santa Cruz S., (2001). A novel strategy for the expression of foreign genes from plant virus-vectors FEBS let 489:215-219.

Wang J, Pichersky E (1998) Characterization of S-adenosyl-L-methionine: (iso)eugenol O-methyltransferase involved in floral scent production in Clarkia breweri. Arch Biochem Biophys 1;349(1): 153-60.

Watanabe N., Watanabe S., Nakajima R., Moon J. H., Shimokihara K., Inagaki F., Etoh H., Asai T., Sakatam K., Ina K. (1993). Formation of flower fragrance compounds from their precursors by enzymatic action during flower opening. Biosci. Biotech. Biochem. 57:1101-1106.

Widrelechner M. P., Senechal N. P. (1992). Relationships between nectar production and honeybee preference. Bee World 73:119-127.

Winston M. (1995). Things that don't work. Bee Culture 123:559-560.

Wolf S., Lensky Y., Paldi N. (1999) Genetic variability in flower attractiveness to honeybees (Apis mellifera L.) within the genus Citrullus. HortSci. 34(5):860-863.

What is claimed is:

1. A method of enhancing insect assisted cross-pollination between flowering plants of a single plant species, the flowering plants being of at least two different genetic backgrounds, the method comprising co-expressing in plants of said at least two different genetic backgrounds at least one scent biosynthetic enzyme and growing the plants in a cross-pollination vicinity in a presence of at least one pollinating insect.

2. The method of claim 1, wherein said at least two different genetic backgrounds are paternal and maternal lines used for hybrid seed production.

3. The method of claim 1, wherein said at least two different genetic backgrounds represent different cultivars.

4. The method of claim 1, wherein said plants of at least two different genetic backgrounds are characterized by producing differential pollinator rewards.

5. The method of claim 4, wherein said differential pollinator rewards include different types of differential pollinator rewards.

6. The method of claim 4, wherein said differential pollinator rewards include different amounts of a single differential pollinator reward.

7. The method of claim 4, wherein said differential pollinator rewards include different amounts of a single differential pollinator reward and different types of differential pollinator rewards.

8. The method of claim 1, wherein said plants of at least two different genetic backgrounds are characterized by producing differential pollinator rewards during at least one given seasonal time period.

9. The method of claim 1, wherein said at least one pollinating insect includes bees.

10. The method of claim 1, wherein said bees are honeybees.

11. The method of claim 1, wherein said bees are bumblebees.

12. The method of claim 1, wherein said at least one pollinating insect is selected from the group consisting of a bee, a beetle, a fly and a moth.

13. The method of claim 1, wherein said at least one pollinating insect is native to an area in which the plants are grown.

14. The method of claim 1, wherein said at least one pollinating insect is man-introduced to an area in which the plants are grown.

15. The method of claim 1, wherein said introduction is via at least one beehive.

16. The method of claim 1, wherein the plants are grown in a field.

17. The method of claim 1, wherein the plants are grown in a greenhouse.

18. The method of claim 1, wherein the plants species is selected from the group consisting of sunflower, cotton, melons, onion, tomatoes, cucumbers, pepper, soya, alfalfa, clover and other plant species in which hybrid seed production is practiced and also in apples, pears, cherries, almonds, kiwi and avocado.

19. The method of claim 1, wherein co-expressing said at least one scent biosynthetic enzyme in said plants of said plants of at least two different genetic backgrounds is to an extent so as to reduce an ability of said pollinating insect to differentiate between flowers of said different genetic backgrounds.

20. The method of claim 1, wherein co-expressing said at least one scent biosynthetic enzyme in said plants of said plants of at least two different genetic backgrounds is effected by transforming or infecting the plants with a vector.

21. The method of claim 20, wherein the vector is a plant virus.

22. The method of claim 21, wherein the plant virus has been modified to restrict a severity of infection symptoms to the plants.

23. The method of claim 21, wherein the plant virus has been modified to restrict a natural transfer by an insect-vector.

24. The method of claim 1, wherein co-expressing said at least one scent biosynthetic enzyme in said plants of said plants of at least two different genetic backgrounds is under a control of a constitutive promoter.

25. The method of claim 1, wherein co-expressing said at least one scent biosynthetic enzyme in said plants of said plants of at least two different genetic backgrounds is under a control of a tissue specific promoter.

26. The method of claim 25, wherein said tissue specific promoter is selected from the group consisting of an epithelial specific promoter, a flower specific promoter and a nectary specific promoter.

27. The method of claim 1, wherein said at least one scent biosynthetic enzyme is selected from the group consisting of a monoterpene synthase, an acetyl transferase and a methyltransferase.

28. The method of claim 1, wherein the cross-pollination between said at least two genetic backgrounds is essential and rudimentary.

29. The method of claim 1, wherein the cross-pollination between said at least two genetic backgrounds is beneficial.

30. A method of enhancing insect assisted cross-pollination between flowering plants of a single plant species, the flowering plants being of at least two different cultivars, the method comprising co-expressing in plants of said at least two different cultivars at least one scent biosynthetic enzyme and growing the plants in a cross-pollination vicinity in a presence of at least one pollinating insect.

31. The method of claim 30, wherein said at least two different cultivars are paternal and maternal lines used for hybrid seed production.

32. The method of claim 30, wherein said at least two different cultivars are characterized by producing differential pollinator rewards.

33. The method of claim 32, wherein said differential pollinator rewards include different types of differential pollinator rewards.

34. The method of claim 32, wherein said differential pollinator rewards include different amounts of a single differential pollinator reward.

35. The method of claim 32, wherein said differential pollinator rewards include different amounts of a single differential pollinator reward and different types of differential pollinator rewards.

36. The method of claim 30, wherein said at least two different cultivars are characterized by producing differential pollinator rewards during at least one given seasonal time period.

37. The method of claim 30, wherein said at least one pollinating insect includes bees.

38. The method of claim 30, wherein said bees are honeybees.

39. The method of claim 30, wherein said bees are bumblebees.

40. The method of claim 30, wherein said at least one pollinating insect is selected from the group consisting of a bee, a beetle, a fly and a moth.

41. The method of claim 30, wherein said at least one pollinating insect is native to an area in which the plants are grown.

42. The method of claim 30, wherein said at least one pollinating insect is man-introduced to an area in which the plants are grown.

43. The method of claim 30, wherein said introduction is via at least one beehive.

44. The method of claim 30, wherein the plants are grown in a field.

45. The method of claim 30, wherein the-plants are grown in a greenhouse.

46. The method of claim 30, wherein the plants species is selected from the group consisting of sunflower, cotton, melons, onion, tomatoes, cucumbers, pepper, soya, alfalfa, clover and other plant species in which hybrid seed production is practiced and also in apples, pears, cherries, almonds, kiwi and avocado.

47. The method of claim 30, wherein co-expressing said at least one scent biosynthetic enzyme in said plants of said at least two different cultivars is to an extent so as to reduce an ability of said pollinating insect to differentiate between said cultivars.

48. The method of claim 30, wherein co-expressing said at least one scent biosynthetic enzyme in said plants of said at least two different cultivars is effected by transforming or infecting the plants with a vector.

49. The method of claim 48, wherein the vector is a plant virus.

50. The method of claim 49, wherein the plant virus has been modified to restrict a severity of infection symptoms to the plants.

51. The method of claim 49, wherein the plant virus has been modified to restrict a natural transfer by an insect-vector.

52. The method of claim 30, wherein co-expressing said at least one scent biosynthetic enzyme in said plants of said at least two different cultivars is under a control of a constitutive promoter.

53. The method of claim 30, wherein co-expressing said at least one scent biosynthetic enzyme in said plants of said at least two different cultivars is under a control of a tissue specific promoter.

54. The method of claim 53, wherein said tissue specific promoter is selected from the group consisting of an epithelial specific promoter, a flower specific promoter and a nectary specific promoter.

55. The method of claim 30, wherein said at least one scent biosynthetic enzyme is selected from the group consisting of a monoterpene synthase, an acetyl transferase and a methyltransferase.

56. The method of claim 30, wherein the cross-pollination between said at least two cultivars is essential and rudimentary.

57. The method of claim 30, wherein the cross-pollination between said at least two cultivars is beneficial.

58. A method of enhancing insect assisted cross-pollination between parental and maternal lines of plants used in hybrid seed production, the method comprising co-expressing in plants of said parental and maternal lines at least one scent biosynthetic enzyme and growing the plants in a cross-pollination vicinity in a presence of at least one pollinating insect.

59. The method of claim 58, wherein said maternal line is male sterile.

60. The method of claim 58, wherein said parental and maternal lines are characterized by producing differential pollinator rewards.

61. The method of claim 60, wherein said differential pollinator rewards include different types of differential pollinator rewards.

62. The method of claim 60, wherein said differential pollinator rewards include different amounts of a single differential pollinator reward.

63. The method of claim 60, wherein said differential pollinator rewards include different amounts of a single differential pollinator reward and different types of differential pollinator rewards.

64. The method of claim 58, wherein said parental and maternal lines are characterized by producing differential pollinator rewards during at least one given seasonal time period.

65. The method of claim 58, wherein said at least one pollinating insect includes bees.

66. The method of claim 58, wherein said bees are honeybees.

67. The method of claim 58, wherein said bees are bumblebees.

68. The method of claim 58, wherein said at least one pollinating insect is selected from the group consisting of a bee, a beetle, a fly and a moth.

69. The method of claim 58, wherein said at least one pollinating insect is native to an area in which the plants are grown.

70. The method of claim 58, wherein said at least one pollinating insect is man-introduced to an area in which the plants are grown.

71. The method of claim 58, wherein said introduction is via at least one beehive.

72. The method of claim 58, wherein the plants are grown in a field.

73. The method of claim 58, wherein the plants are grown in a greenhouse.

74. The method of claim 58, wherein the plants species is selected from the group consisting of sunflower, cotton, melons, onion, tomatoes, cucumbers, pepper, soya, alfalfa, clover and other plant species in which hybrid seed production is practiced and also in apples, pears, cherries, almonds, kiwi and avocado.

75. The method of claim 58, wherein co-expressing said at least one scent biosynthetic enzyme in said plants parental and maternal lines is to an extent so as to reduce an ability of said pollinating insect to differentiate between plants of said parental and maternal lines.

76. The method of claim 58, wherein co-expressing said at least one scent biosynthetic enzyme in said plants of said parental and maternal lines is effected by transforming or infecting the plants with a vector.

77. The method of claim 76, wherein the vector is a plant virus.

78. The method of-claim-77, wherein the plant virus has been modified to restrict a severity of infection symptoms to the plants.

79. The method of claim 77, wherein the plant virus has been modified to restrict a natural transfer by an insect-vector.

80. The method of claim 58, wherein co-expressing said at least one scent biosynthetic enzyme in said plants of said parental and maternal lines is under a control of a constitutive promoter.

81. The method of claim 58, wherein co-expressing said at least one scent biosynthetic enzyme in said plants of said parental and maternal lines is under a control of a tissue specific promoter.

82. The method of claim 81, wherein said tissue specific promoter is selected from the group consisting of an epithelial specific promoter, a flower specific promoter and a nectary specific promoter.

83. The method of claim 58, wherein said at least one scent biosynthetic enzyme is selected from the group consisting of a monoterpene synthase, an acetyl transferase and a methyltransferase.

84. A method of reducing associative learning of a pollinating insect, the method comprising exposing said pollinating insect to at least two differential pollinator rewards, each of said at least two differential pollinator rewards being scented with an identical scent.

85. The method of claim 84, wherein exposing said pollinating insect to at least two differential pollinator rewards is effected by allowing said pollinating insects to feed on flowering plants of a single plant species, the flowering plants producing said at least two differential pollinator rewards, and the flowering plants co-producing at least one scent biosynthetic enzyme and are therefore scented with said identical scent.
